(19) United States
(12) Patent Application Publication
(10) Pub. No.: US 2022/0228186 A1
(43) Pub. Date: Jul. 21, 2022

(54) COMPOSITIONS AND METHODS FOR PRODUCING STEVIOL GLYCOSIDES

(71) Applicant: Arzeda Corp., Seattle, WA (US)

(72) Inventors: Alexandre ZANGHELLINI, Seattle, WA (US); Kyle ROBERTS, Seattle, WA (US); Mark NANCE, Seattle, WA (US); Joshua RHEA, Seattle, WA (US)

(21) Appl. No.: 17/613,752

(22) PCT Filed: May 22, 2020
(86) PCT No.: PCT/US2020/034417

§ 371 Date: Nov. 23, 2021

Related U.S. Application Data
(60) Provisional application No. 62/851,772, filed on May 23, 2019.

(51) Int. Cl. C12P 19/56; C12P 19/18; C12N 9/10; C12N 1/20; C12N 15/70; C07H 15/24

(52) U.S. Cl. CPC C12P 19/56; C12P 19/18; C12N 9/1051; C12N 2800/101; C12N 15/70; C07H 15/24; C12N 1/20

(57) ABSTRACT

The present invention provides compositions and methods for producing steviol glycosides, particularly rebD, rebM and isomers thereof. The method includes use of glycosyltransferases that can transfer a glucose moiety from non-UDP-sugar sugar donors to steviol glycosides.

### CROSS-REFERENCE TO RELATED APPLICATIONS

This application claims the benefit of priority to U.S. provisional application No. 62/851,772 filed on May 23, 2019, which is hereby incorporated by reference in its entirety.

### CROSS-REFERENCE TO SEQUENCE LISTING

The contents of the text file submitted electronically herewith are incorporated herein by reference in their entirety: A computer readable format copy of the Sequence Listing (file-name: ARZE_030_01WO_SeqList_ST25.txt, date recorded: May 20, 2020, file size 689 kilo-bytes).

### FIELD

The present disclosure relates to enzymes and biocatalytic processes for producing steviol glycosides. The present disclosure particularly relates to use of glycosyltransferases that can transfer a glucose moiety from non-UDP-sugar sugar donors to steviol glycosides.

### BACKGROUND

The species Stevia rebaudiana is commonly grown for its sweet leaves, which have traditionally been used as a sweetener. Stevia extract is considered 200-300 times sweeter than sugar and is used commercially as a high intensity sweetener. The main glycoside component of stevia leaf are steviosides and rebaudiosides. Over ten different steviol glycosides are present in appreciable quantities in the leaf. The principal sweetening compounds are stevioside and rebaudioside A. Rebaudioside A (Reb A) is considered higher value compared to stevioside, because of its increased sweetness and decreased bitterness.

### SUMMARY OF THE INVENTION

The present disclosure provides a method for transferring a sugar moiety to a substrate steviol glycoside.

The sweetness and bitterness profiles of Reb D and Reb M are improved compared to Reb A, but are present at very low quantities in the stevia leaf. Reb D can be made by the addition of a single glucose molecule to Reb A. Similarly, Reb M can be produced by adding a single glucose molecule to Reb D. Native glycosyltransferases that make Reb D and Reb M use UDP-glucose as the glucose source for transferring to Reb A or Reb D, respectively. However, UDP-glucose is an expensive co-substrate and adds significant costs to any process that utilizes the compound. In some embodiments, the method comprises contacting the substrate steviol glycoside with a glycosyltransferase polypeptide and a non-UDP-sugar sugar donor.

Advantageously, the methods provide great economic benefit to making these rebaudiosides through an enzymatic reaction that uses an alternative, less expensive sugar donor than UDP-glucose. Thus, the disclosed methods provide novel glycosyltransferases and methods for transferring a glucose moiety from non-UDP-sugar sugar donors to steviol glycosides.

The methods of the disclosure provide glycosyltransferase polypeptides that comprise an amino acid sequence that is at least 60%, at least 65%, at least 70%, at least 75%, at least 80%, at least 85%, at least 90%, at least 95%, at least 98%, or at least 99% identical to an amino acid sequence selected from the group consisting of SEQ ID NOs: 1-128. The glycosyltransferase polypeptide may comprise, or consist of, an amino acid sequence selected from the group consisting of SEQ ID NOs: 1-128. In aspects, more than one polypeptide may be used; for example, SEQ ID NOs: 121 and 128 for a heterodimer and may be used together in methods and compositions disclosed herein.

In some embodiments, the substrate steviol glycoside is rebaudioside A, or isomers thereof, and the glycosyltransferase polypeptide comprises, or consists of, an amino acid sequence selected from the group consisting of SEQ ID NOs: 1, 19, 29, 32, 41, 42, 43, 44, 45, 46, 47, 48, 49, 50, 51, 52, 53, 54, 55, 56, 58, 61, 65, 67, 68, 81, 87, 90, 91, 92, 93, 94, 95, 96, 97, 98, 99, 100, 101, 102, 105, 106, 116, 120, 122, and 127, and combinations thereof.

In some embodiments, the substrate steviol glycoside is rebaudioside D, or isomers thereof, and the glycosyltransferase polypeptide comprises, or consists of, an amino acid sequence selected from the group consisting of SEQ ID NOs: 2, 3, 22, 29, 39, 41, 42, 45, 49, 50, 51, 52, 53, 54, 55, 56, 62, 63, 72, 87, 90, 91, 94, 97, 98, 99, 100, 101, 102, 109, 110, 111, 112, 113, 115, 117, 118, 119, 121, 123, 124, 125, 126, 128, and combinations thereof.

In other embodiments, the substrate steviol glycoside is steviol, steviol-13-O-glucoside, steviol-19-O-glucoside, rubusoside, steviol-1,2-bioside, steviol-1,3-bioside, rubusoside, dulcoside B, dulcoside A, rebaudioside B, rebaudioside G, stevioside, rebaudioside C, rebaudioside F, rebaudioside A, rebaudioside I, rebaudioside E, rebaudioside H, rebaudioside L, rebaudioside K, rebaudioside J, rebaudioside M, rebaudioside D, rebaudioside N, rebaudioside O, rebaudioside Q, an isomer thereof, a synthetic steviol glycoside or combinations thereof.

In further embodiments, the non-UDP-sugar sugar donor is alpha-glucose-1-phosphate, beta-glucose-1-phosphate, cellobiose, sucrose, maltose, gentiobiose, trehalose, kojibiose, nigerose, isomaltose, beta-beta-trehalose, alpha-beta-trehalose, sophorose, laminaribiose, turanose, maltulose, palatinose, gentiobiulose, nigerotriose, maltotriose, melezitose, maltotriulose, kestose, starch, cellulose, glycogen, amylose, amylopectin, dextran, dextrin, maltodextrin, glucose syrup, cellodextrin, cyclodextrin, a non-UDP nucleotide sugar (e.g., ADP-glucose, GDP-glucose, CDP-glucose, TDP-glucose), or a steviol glycoside.

The present disclosure also provides a method for producing a target steviol glycoside composition, comprising the steps of: (a) providing a starting composition comprising greater than about 0.5%, about 1%, about 2%, about 3%, about 4%, about 5%, about 10%, about 20%, about 30%, about 40%, about 50%, about 60%, about 70%, about 80%, about 90%, about 92%, about 93%, about 94%, about 95%, about 96%, about 97%, about 98%, about 99%, or about 99.6% of a substrate steviol glycoside by weight on an anhydrous basis; (b) contacting the starting composition with a glycosyltransferase polypeptide and a non-UDP-sugar sugar donor; and (c) producing a target composition comprising a target steviol glycoside.

The method of the present disclosure teaches that the glycosyltransferase polypeptide comprises an amino acid sequence that is at least 60%, at least 65%, at least 70%, at least 75%, at least 80%, at least 85%, at least 90%, at least 95%, at least 98%, or at least 99% identical to an amino acid sequence selected from the group consisting of SEQ ID NOs: 1-128. The method of the present disclosure teaches the glycosyltransferase polypeptide comprises an amino acid sequence selected from the group consisting of SEQ ID NOs: 1-128.

In some embodiments, the target composition comprises greater than about 0.5%, about 1%, about 2%, about 3%, about 4%, about 5%, about 10%, about 20%, about 30%, about 40%, about 50%, about 60%, about 70%, about 80%, about 85%, about 90%, about 91%, about 92%, about 93%, about 94%, about 95%, about 96%, about 97%, about 98%, about 99%, about 99.6% of the target steviol glycoside by weight on an anhydrous basis.

In embodiments, the substrate steviol glycoside is rebaudioside A, or isomers thereof; and the target steviol glycoside is rebaudioside D, or isomers thereof. For example, the target steviol glycoside is a rebaudioside D isomer. In embodiments, the rebaudioside D isomer is rebaudioside D_1.07, rebaudioside D_0.77, rebaudioside D_1.21, rebaudioside D_1.25, or rebaudioside D_1.29. In embodiments, the target steviol glycoside is a rebaudioside M isomer. In further embodiments, the rebaudioside M isomer is rebaudioside M_0.66, rebaudioside M_0.80, rebaudioside M_0.92, rebaudioside M_1.06, rebaudioside M_1.11, rebaudioside M_1.17, or rebaudioside M_1.51. In other embodiments, the target steviol glycoside is a rebaudioside with 7 glucose moieties (rebaudioside M plus 1 glucose isomer; rebaudioside Mp1 isomer; rebMp1 isomer). In further embodiments, the rebaudioside Mp1 isomer is rebaudioside Mp1_0.62, rebaudioside Mp1_0.76, rebaudioside Mp1_0.82, rebaudioside Mp1_0.88, rebaudioside Mp1_0.94, rebaudioside Mp1_1.09, rebaudioside Mp1_1.14, rebaudioside Mp1_1.19, rebaudioside Mp1_1.27, rebaudioside Mp1_1.42, or rebaudioside Mp1_1.66.

In some embodiments, the substrate steviol glycoside is rebaudioside D, or isomers thereof; and the target steviol glycoside is rebaudioside M, or isomers thereof. In some embodiments, the target steviol glycoside is a rebaudioside M isomer. In some embodiments, the rebaudioside M isomer is rebaudioside M_0.66, rebaudioside M_0.80, rebaudioside M_0.92, rebaudioside M_1.06, rebaudioside M_1.11, rebaudioside M_1.17, or rebaudioside M_ 1.51. In other embodiments, the target steviol glycoside is a rebaudioside with 7 glucose moieties (rebaudioside M plus 1 glucose isomer; rebaudioside Mp1 isomer). In further embodiments, the rebaudioside Mp1 isomer is rebaudioside Mp1_0.62, rebaudioside Mp1_0.76, rebaudioside Mp1_0.82, rebaudioside Mp1_0.88, rebaudioside Mp1_0.94, rebaudioside Mp1_1.09, rebaudioside Mp1_1.14, rebaudioside Mp1_1.19, rebaudioside Mp1_1.27, rebaudioside Mp1_1.42, rebaudioside Mp1_1.66.

The present disclosure also provides a method for producing a target steviol glycoside composition, comprising the steps of: (a) providing a starting composition comprising greater than about 0.5%, about 1%, about 2%, about 3%, about 4%, about 5%, about 10%, about 20%, about 30%, about 40%, about 50%, about 60%, about 70%, about 80%, about 90%, about 92%, about 93%, about 94%, about 95%, about 96%, about 97%, about 98%, about 99%, or about 99.6% of a substrate steviol glycoside by weight on an anhydrous basis; (b) contacting the starting composition with a first glycosyltransferase polypeptide and a non-UDP-sugar sugar donor; (c) producing an intermediate composition comprising an intermediate target steviol glycoside; (d) contacting the intermediate composition with a second glycosyltransferase polypeptide and the non-UDP-sugar sugar donor; and (e) producing a target composition comprising a target steviol glycoside.

The method of the present disclosure teaches that the first glycosyltransferase polypeptide comprises an amino acid sequence that is at least 60%, at least 65%, at least 70%, at least 75%, at least 80%, at least 85%, at least 90%, at least 95%, at least 98%, or at least 99% identical to an amino acid sequence selected from the group consisting of SEQ ID NOs: 1-128. In some embodiments, the second glycosyltransferase polypeptide comprises an amino acid sequence that is at least 60%, at least 65%, at least 70%, at least 75%, at least 80%, at least 85%, at least 90%, at least 95%, at least 98%, or at least 99% identical to an amino acid sequence selected from the group consisting of SEQ ID NOs: 1-128. In some embodiments, the first glycosyltransferase polypeptide and the second glycosyltransferase polypeptide are identical. In some embodiments, the first glycosyltransferase polypeptide and the second glycosyltransferase polypeptide are different.

The present disclosure also provides a recombinant glycosyltransferase polypeptide comprising an amino acid sequence that is at least 60%, at least 65%, at least 70%, at least 75%, at least 80%, at least 85%, at least 90%, at least 95%, at least 98%, or at least 99% identical to an amino acid sequence selected from the group consisting of SEQ ID NOs: 1-128, or combinations thereof. The recombinant glycosyltransferase polypeptide may comprises an amino acid sequence that is at least 60% identical to an amino acid sequence selected from the group consisting of SEQ ID NOs: 2, 3, 22, 29, 39, 41, 42, 45, 49, 50, 51, 52, 53, 54, 55, 56, 62, 63, 72, 87, 90, 91, 94, 97, 98, 99, 100, 101, 102, 109, 110, 111, 112, 113, 115, 117, 118, 119, 121, 123, 124, 125, 126, and 128. In some embodiments, the recombinant glycosyltransferase polypeptide comprises an amino acid sequence that is at least 60% identical to an amino acid sequence selected from the group consisting of SEQ ID NOs: 1, 19, 29, 32, 41, 42, 43, 44, 45, 46, 47, 48, 49, 50, 51, 52, 53, 54, 55, 56, 58, 61, 65, 67, 68, 81, 87, 90, 91, 92, 93, 94, 95, 96, 97, 98, 99, 100, 101, 102, 105, 106, 116, 120, 122, and 127.

In some embodiments, the glycosyltransferase polypeptide comprises an amino acid sequence selected from the group consisting of SEQ ID NOs: 1-128. In other embodiments, the glycosyltransferase polypeptide comprises, or consists of, an amino acid sequence selected from the group consisting of SEQ ID NOs: 1, 19, 29, 32, 41, 42, 43, 44, 45, 46, 47, 48, 49, 50, 51, 52, 53, 54, 55, 56, 58, 61, 65, 67, 68, 81, 87, 90, 91, 92, 93, 94, 95, 96, 97, 98, 99, 100, 101, 102, 105, 106, 116, 120, 122, and 127. In other embodiments, the glycosyltransferase polypeptide comprises, or consists of, an amino acid sequence selected from the group consisting SEQ ID NOs: 2, 3, 22, 29, 39, 41, 42, 45, 49, 50, 51, 52, 53, 54, 55, 56, 62, 63, 72, 87, 90, 91, 94, 97, 98, 99, 100, 101, 102, 109, 110, 111, 112, 113, 115, 117, 118, 119, 121, 123, 124, 125, 126, and 128. In further embodiments, the glycosyltransferase polypeptide is capable of transferring a sugar moiety to a substrate steviol glycoside.

The present disclosure further provides a modified microorganism expressing a glycosyltransferase polypeptide comprises, or consists of, an amino acid sequence that is at least 60%, at least 65%, at least 70%, at least 75%, at least 80%, at least 85%, at least 90%, at least 95%, at least 98%, or at least 99% identical to an amino acid sequence selected from the group consisting of SEQ ID NOs: 1-128. The modified microorganism of the present disclosure teaches that the glycosyltransferase polypeptide comprises, or consists of, an amino acid sequence that is at least 60% identical to an amino acid sequence selected from the group consisting of SEQ ID NOs: 2, 3, 22, 29, 39, 41, 42, 45, 49, 50, 51, 52, 53, 54, 55, 56, 62, 63, 72, 87, 90, 91, 94, 97, 98, 99, 100, 101, 102, 109, 110, 111, 112, 113, 115, 117, 118, 119, 121, 123, 124, 125, 126, and 128. In some embodiments, the glycosyltransferase polypeptide comprises, or consists of, an amino acid sequence selected from the group consisting of SEQ ID NOs: 1-128. In other embodiments, the glycosyltransferase polypeptide comprises, or consists of, an amino acid sequence selected from the group consisting of SEQ ID NOs: 2, 3, 22, 29, 39, 41, 42, 45, 49, 50, 51, 52, 53, 54, 55, 56, 62, 63, 72, 87, 90, 91, 94, 97, 98, 99, 100, 101, 102, 109, 110, 111, 112, 113, 115, 117, 118, 119, 121, 123, 124, 125, 126, and 128. In further embodiments, the glycosyltransferase polypeptide is capable of transferring a sugar moiety to a substrate steviol glycoside.

### BRIEF DESCRIPTION OF THE DRAWINGS

The accompanying drawings are included to provide a further understanding of the disclosure. The drawings illustrate embodiments of the disclosure and together with the description serve to explain the principles of the embodiments of the disclosure.

FIG. 1 shows the conversion (glycosylation) of rebaudioside A (rebA) to rebaudioside D (rebD).

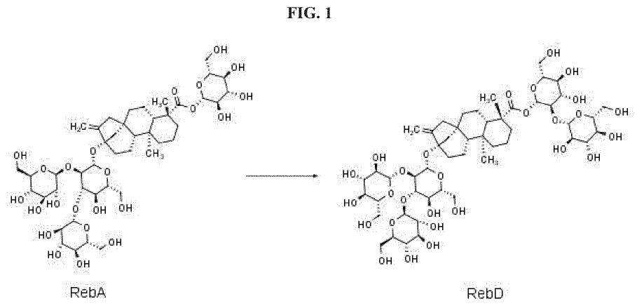

FIG. 2 shows the conversion (glycosylation) of rebD to rebaudioside M (rebM).

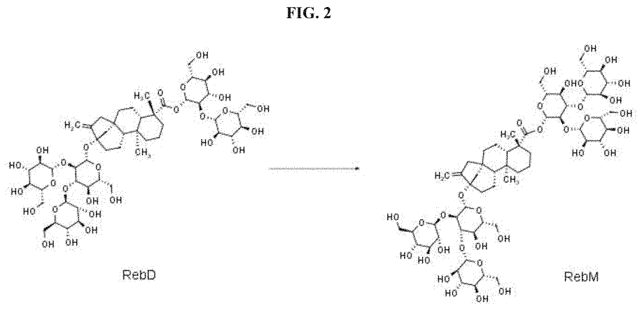

FIG. 3 shows an LC/MS trace of an enzyme that converts rebA to rebD_0.77. In this figure, the glucosyltransferase is used to glycosylate rebA to rebD_0.77 using maltose as the sugar donor.

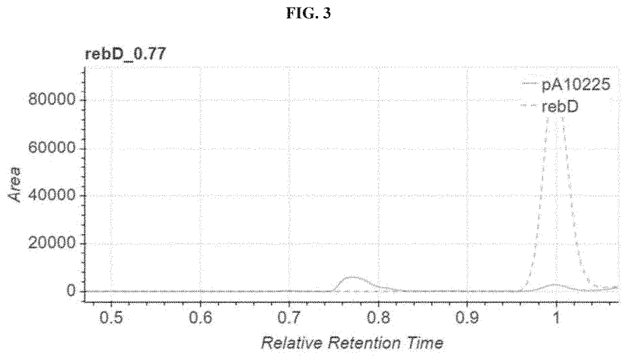

FIG. 4 shows an LC/MS trace of an enzyme that converts rebA to rebD_1.07. In this figure, the glucosyltransferase is used to glycosylate rebA to rebD_1.07 using alpha-glucose1-phosphate as the sugar donor.

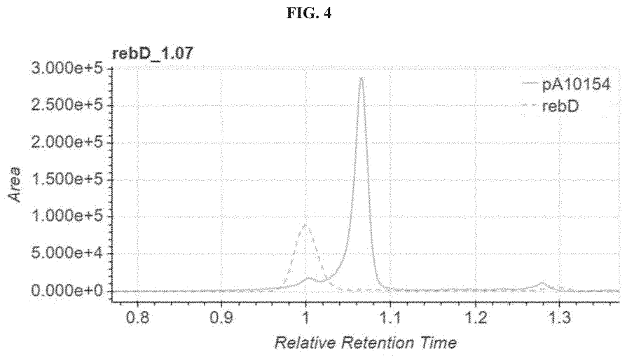

FIG. 5 shows an LC/MS trace of an enzyme that converts rebA to rebD_1.21. In this figure, the glucosyltransferase is used to glycosylate rebA to rebD_1.21 using maltose as the sugar donor.

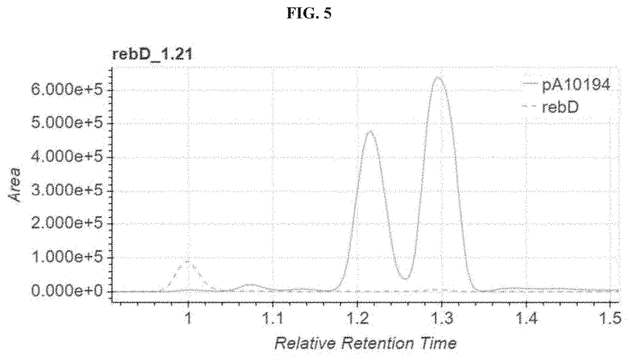

FIG. 6 shows an LC/MS trace of an enzyme that converts rebA to rebD_1.25. In this figure, the glucosyltransferase is used to glycosylate rebA to rebD_1.25 using maltose as the sugar donor.

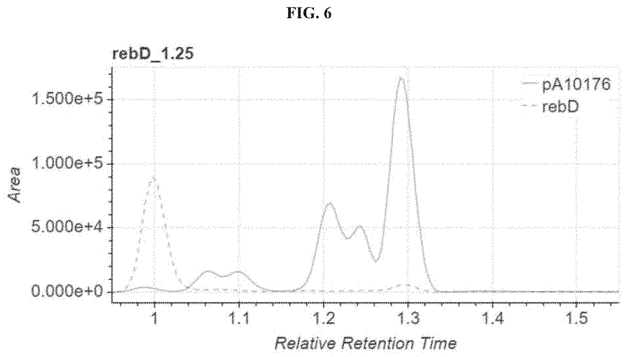

FIG. 7 shows an LC/MS trace of an enzyme that converts rebA to rebD_1.29. In this figure, the glucosyltransferase is used to glycosylate rebA to rebD_1.29 using maltose as the sugar donor.

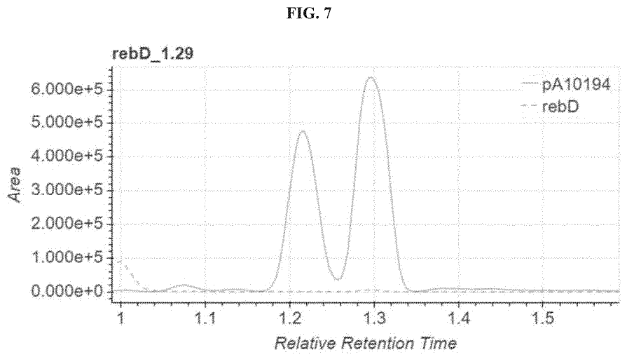

FIG. 8 shows an LC/MS trace of an enzyme that converts rebA to rebM_0.66. In this figure, the glucosyltransferase is used to glycosylate rebA to rebM_0.66 using maltose as the sugar donor.

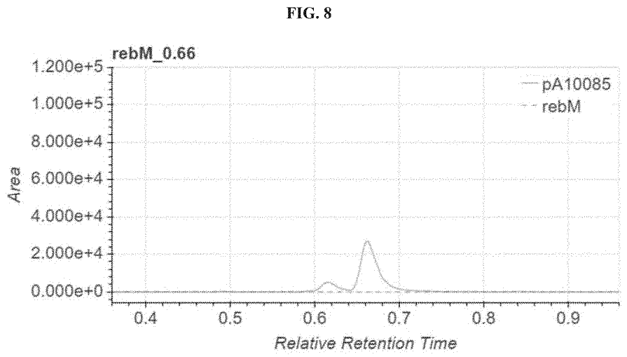

FIG. 9 shows an LC/MS trace of an enzyme that converts rebD to rebM_0.80. In this figure, the glucosyltransferase is used to glycosylate rebD to rebM_0.80 using maltose as the sugar donor.

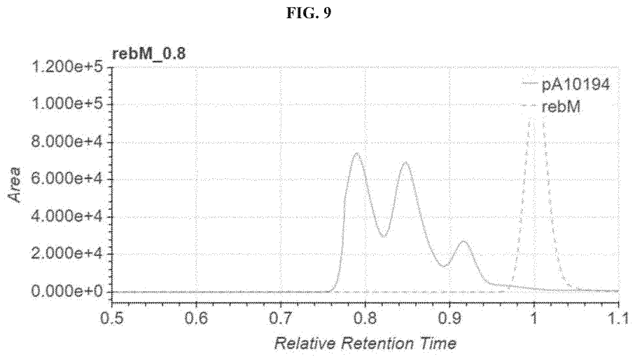

FIG. 10 shows an LC/MS trace of an enzyme that converts rebA to rebM_0.92. In this figure, the glucosyltransferase is used to glycosylate rebA to rebM_0.92 using sucrose as the sugar donor.

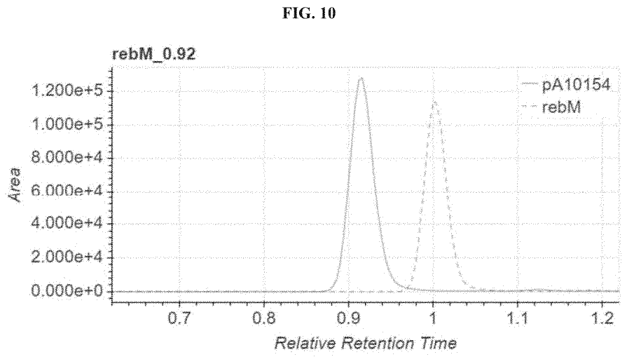

FIG. 11 shows an LC/MS trace of an enzyme that converts rebA to rebM_1.06. In this figure, the glucosyltransferase is used to glycosylate rebA to rebM_1.06 using maltose as the sugar donor.

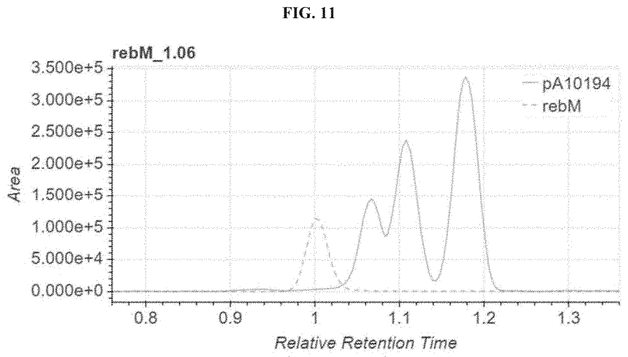

FIG. 12 shows an LC/MS trace of an enzyme that converts rebA to rebM_1.11. In this figure, the glucosyltransferase is used to glycosylate rebA to rebM_1.11 using maltose as the sugar donor.

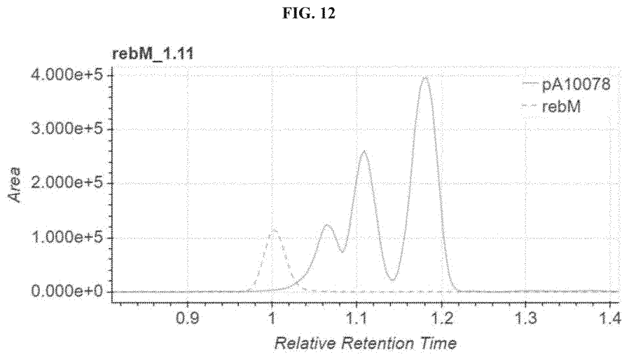

FIG. 13 shows an LC/MS trace of an enzyme that converts rebA to rebM_1.17. In this figure, the glucosyltransferase is used to glycosylate rebA to rebM_1.17 using maltose as the sugar donor.

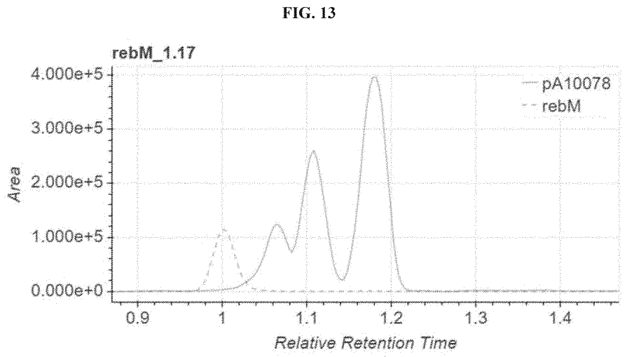

FIG. 14 shows an LC/MS trace of an enzyme that converts rebA to rebM_1.51. In this figure, the glucosyltransferase is used to glycosylate rebA to rebM_1.51 using gentiobiose as the sugar donor.

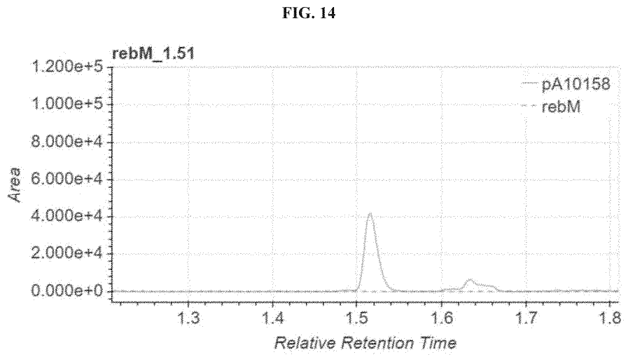

FIG. 15 shows the production of RebD_1.29 by contacting the polypeptide encoded in pA10147 (SEQ NO: 55) with RebA.

FIG. 16 shows the production of RebM by contacting the polypeptide encoded in pA10147 (SEQ NO: 55) with RebD.

FIG. 17 shows the production of RebD_1.07 by contacting the polypeptide encoded in pA10154 (SEQ NO: 42) with increasing concentration of RA50 feed.

FIG. 18 shows the production of RebM_0.66, RebM_0.80 and RebM_0.92 from RebD by several discovered glycosyltransferases.

### DETAILED DESCRIPTION

The present disclosure provides enzymes and a biocatalytic process for preparing a composition comprising a target steviol glycoside by contacting a starting composition comprising a substrate steviol glycoside with a non-UDP-sugar glucosyltransferase polypeptide, thereby producing a composition comprising a target steviol glycoside comprising one or more additional glucose units than the substrate steviol glycoside.

The starting composition can be any composition comprising at least one substrate steviol glycoside. In one embodiment, the substrate steviol glycoside is selected from the group consisting of steviol, steviol-13-O-glucoside, steviol-19-O-glucoside, rubusoside, steviol-1,2bioside, steviol-1,3-bioside, rubusoside, dulcoside B, dulcoside A, rebaudioside B, rebaudioside G, stevioside, rebaudioside C, rebaudioside F, rebaudioside A, rebaudioside I, rebaudioside E, rebaudioside H, rebaudioside L, rebaudioside K, rebaudioside J, rebaudioside M, rebaudioside D, rebaudioside N, rebaudioside O, rebaudioside Q, an isomer thereof, a synthetic steviol glycoside or combinations thereof. The starting composition may be commercially available or prepared. The starting composition may comprise a purified substrate steviol glycoside or a partially purified steviol glycoside substrate.

In one embodiment, the substrate steviol glycoside is stevioside. In another embodiment, the substrate steviol glycoside is rebaudioside A, or isomers thereof. In still another embodiment, the substrate steviol glycoside is rebaudioside D, or isomers thereof.

The target steviol glycoside can be any known steviol glycoside. In one embodiment, the target steviol glycoside is steviol-13-O-glucoside, steviol-19-O-glucoside, rubusoside, steviol-1,2-bioside, steviol-1,3-bioside, rubusoside, dulcoside B, dulcoside A, rebaudioside B, rebaudioside G, stevioside, rebaudioside C, rebaudioside F, rebaudioside A, rebaudioside I, rebaudioside E, rebaudioside H, rebaudioside L, rebaudioside K, rebaudioside J, rebaudioside M, rebaudioside D, rebaudioside N, rebaudioside O, rebaudioside Q, a rebaudioside with 7 covalently attached glucose units (e.g. rebaudioside M plus 1 glucose unit), a synthetic steviol glycoside, an isomer thereof, and/or a steviol glycoside composition.

In embodiments, the target steviol glycoside is rebaudioside A, or isomers thereof, rebaudioside D, or isomers thereof, or rebaudioside M, or isomers thereof. Rebaudioside Mp1 (“M plus one”) are obtained by adding a glucose unit to rebaudioside M or a rebaudioside M isomer.

Rebaudioside isomers may be defined and identified according to their relative retention time compared to a rebaudioside standard. The relative retention time is calculated by dividing the absolute retention time of the isomer by the absolute retention time of the standard rebaudioside molecule. The isomer rebaudioside D_1.29 has a relative retention time of 1.29 compared to rebaudioside D. Similarly, the isomer rebaudioside M_0.66 has a relative retention time of 0.66 compared to the rebaudioside M retention time. The isomer rebaudioside Mp1_0.62 has a relative retention time of 0.62 compared to the rebaudioside D standard. Experimental conditions for identifying rebaudioside D isomers, rebaudioside M isomers and rebaudioside Mp1 isomers are provided in Example 4 and Example 16.

The intermediate and target rebaudioside isomers in methods and compositions disclosed herein include one or more of rebaudioside D_0.77, rebaudioside D_1.07, rebaudioside D_1.21, rebaudioside D_1.25, rebaudioside D_1.29, rebaudioside M_0.66, rebaudioside M_0.80, rebaudioside M_0.92, rebaudioside M_1.06, rebaudioside M_1.11, rebaudioside M_1.17, rebaudioside M_1.51, rebaudioside Mp1_0.62, rebaudioside Mp1_0.76, rebaudioside Mp1_0.82, rebaudioside Mp1_0.88, rebaudioside Mp1_0.94, rebaudioside Mp1_1.09, rebaudioside Mp1_1.14, rebaudioside Mp1_1.19, rebaudioside Mp1_1.27, rebaudioside Mp1_1.42, rebaudioside Mp1_1.66.

The glycosyltransferase polypeptide can be any glucosyltransferase capable of adding at least one glucose unit to the substrate steviol glycoside to provide the target steviol glycoside using a non-UDP-sugar sugar source. In one embodiment, the non-UDP-sugar glucosyltransferase polypeptide is expressed in a host microorganism. The host may be, for example, E. coli, Saccharomyces sp., Aspergillus sp., Pichia sp., Bacillus sp. In another embodiment, the glycosyltransferase polypeptide is synthesized.

The glycosyltransferase polypeptide can be provided in any suitable form, including free, immobilized or as a whole cell system. The degree of purity of the glucosyltransferase polypeptide may vary, e.g., it may be provided as a crude, semi-purified or purified enzyme preparation(s).

In one embodiment, the glycosyltransferase polypeptide is free. In another embodiment, the glycosyltransferase polypeptide is immobilized to a solid support, for example on an inorganic or organic support. In some embodiments, the solid support is derivatized cellulose, glass, ceramic, methacrylate, styrene, acrylic, a metal oxide, or a membrane. In some embodiments, the glucosyltransferase polypeptide is immobilized to the solid support by covalent attachment, adsorption, cross-linking, entrapment, or encapsulation.

In yet another embodiment, the glycosyltransferase polypeptide is provided in the form of a whole cell system, for example as a living fermentative microbial cell, or as dead and stabilized microbial cell, or in the form of a cell lysate.

In one embodiment, the glucosyltransferase polypeptide is any glucosyltransferase capable of adding at least one glucose unit to rebA to form rebD or a rebD isomer using a non-UDP-sugar sugar donor. In a particular embodiment, the glucosyltransferase polypeptide is one of SEQ ID NOs: 1, 19, 29, 32, 41, 42, 43, 44, 45, 46, 47, 48, 49, 50, 51, 52, 53, 54, 55, 56, 58, 61, 65, 67, 68, 81, 87, 90, 91, 92, 93, 94, 95, 96, 97, 98, 99, 100, 101, 102, 105, 106, 116, 120, 122, and 127. In another embodiment, the glucosyltransferase polypeptide is a polypeptide sequence that is at least 60%, 65%, 70%, 75%, 80%, 85%, 86%, 87%, 88%, 89%, 90%, 91%, 92%, 93%, 94%, 95%, 96%, 97%, 98%, 99%, or more sequence identity to one of SEQ ID NOs: 1, 19, 29, 32, 41, 42, 43, 44, 45, 46, 47, 48, 49, 50, 51, 52, 53, 54, 55, 56, 58, 61, 65, 67, 68, 81, 87, 90, 91, 92, 93, 94, 95, 96, 97, 98, 99, 100, 101, 102, 105, 106, 116, 120, 122, and 127.

Percentage identity may be calculated using the alignment program Clustal Omega (available at /www.ebi.ac.uk/Tools/msa/clustalo/) default settings. The default transition matrix is Gonnet, gap opening penalty is 6 bits, gap extension is 1 bit. Clustal Omega uses the HHalign algorithm and its default settings as its core alignment engine. The algorithm is described in Soding, J. (2005) ‘Protein homology detection by HMM-HMM comparison’. Bioinformatics 21, 951-960.

In one embodiment, the glucosyltransferase polypeptide is any glucosyltransferase capable of adding at least one glucose unit to rebD or a rebD isomer to form rebM or a rebM isomer using a non-UDP-sugar sugar donor. In a particular embodiment, the glucosyltransferase polypeptide is one of SEQ ID NOs: 2, 3, 22, 29, 39, 41, 42, 45, 49, 50, 51, 52, 53, 54, 55, 56, 62, 63, 72, 87, 90, 91, 94, 97, 98, 99, 100, 101, 102, 109, 110, 111, 112, 113, 115, 117, 118, 119, 121, 123, 124, 125, 126, and 128. In another embodiment, the glucosyltransferase polypeptide is a polypeptide sequence that is at least 60%, 65%, 70%, 75%, 80%, 85%, 86%, 87%, 88%, 89%, 90%, 91%, 92%, 93%, 94%, 95%, 96%, 97%, 98%, 99%, or more sequence identity to one of SEQ ID NOs: SEQ ID NOs: 2, 3, 22, 29, 39, 41, 42, 45, 49, 50, 51, 52, 53, 54, 55, 56, 62, 63, 72, 87, 90, 91, 94, 97, 98, 99, 100, 101, 102, 109, 110, 111, 112, 113, 115, 117, 118, 119, 121, 123, 124, 125, 126, and 128.

Plasmids containing nucleic acids encoding enzymes having SEQ ID NOS:1-128 are described in the table below.

| Plasmid ID | SEQ ID NO: |
|---|---|
| pA10225 | 1 |
| pA10143 | 2 |
| pA10080 | 3 |
| pA10135 | 4 |
| pA10139 | 5 |
| pA10170 | 6 |
| pA10122 | 7 |
| pA10260 | 8 |
| pA10113 | 9 |
| pA10180 | 10 |
| pA10163 | 11 |
| pA10141 | 12 |
| pA10181 | 13 |
| pA10231 | 14 |
| pA10243 | 15 |
| pA10172 | 16 |
| pA10108 | 17 |
| pA10263 | 18 |
| pA10251 | 19 |
| pA10118 | 20 |
| pA10223 | 21 |
| pA10082 | 22 |
| pA10157 | 23 |
| pA10206 | 24 |
| pA10166 | 25 |
| pA10192 | 26 |
| pA10081 | 27 |
| pA10204 | 28 |
| pA10085 | 29 |
| pA10177 | 30 |
| pA10121 | 31 |
| pA10105 | 32 |
| pA10209 | 33 |
| pA10216 | 34 |
| pA10123 | 35 |
| pA10221 | 36 |
| pA10140 | 37 |
| pA10134 | 38 |
| pA10109 | 39 |
| pA10131 | 40 |
| pA10174 | 41 |
| pA10154 | 42 |
| pA10152 | 43 |
| pA10151 | 44 |
| pA10076 | 45 |
| pA10155 | 46 |
| pA10072 | 47 |
| pA10265 | 48 |
| pA10074 | 49 |
| pA10075 | 50 |
| pA10194 | 51 |
| pA10176 | 52 |
| pA10078 | 53 |
| pA10073 | 54 |
| pA10147 | 55 |
| pA10158 | 56 |
| pA10116 | 57 |
| pA10262 | 58 |
| pA10203 | 59 |
| pA10266 | 60 |
| pA10149 | 61 |
| pA10197 | 62 |
| pA10195 | 63 |
| pA10212 | 64 |
| pA10115 | 65 |
| pA10211 | 66 |
| pA12546 | 67 |
| pA10114 | 68 |
| pA10162 | 69 |
| pA10126 | 70 |
| pA10125 | 71 |
| pA10080 | 72 |
| pA10122 | 73 |
| pA10260 | 74 |
| pA10113 | 75 |
| pA10163 | 76 |
| pA10181 | 77 |
| pA10231 | 78 |
| pA10243 | 79 |
| pA10263 | 80 |
| pA10251 | 81 |
| pA10118 | 82 |
| pA10157 | 83 |
| pA10206 | 84 |
| pA10166 | 85 |
| pA10204 | 86 |
| pA10085 | 87 |
| pA10209 | 88 |
| pA10123 | 89 |
| pA10174 | 90 |
| pA10154 | 91 |
| pA10152 | 92 |
| pA10151 | 93 |
| pA10076 | 94 |
| pA10155 | 95 |
| pA10072 | 96 |
| pA10074 | 97 |
| pA10075 | 98 |
| pA10194 | 99 |
| pA10176 | 100 |
| pA10078 | 101 |
| pA10073 | 102 |
| pA10203 | 103 |
| pA10266 | 104 |
| pA10149 | 105 |
| pA10115 | 106 |
| pA10162 | 107 |
| pA10125 | 108 |
| pA10112 | 109 |
| pA10213 | 110 |
| pA10175 | 111 |
| pA10117 | 112 |
| pA10259 | 113 |
| pA10190 | 114 |
| pA10098 | 115 |
| pA10188 | 116 |
| pA10189 | 117 |
| pA10160 | 118 |
| pA10084 | 119 |
| pA10185 | 120 |
| pA10273* | 121 |
| pA10224 | 122 |
| pA10175 | 123 |
| pA10259 | 124 |
| pA10098 | 125 |
| pA10160 | 126 |
| pA10224 | 127 |
| pA10273* | 128 |

*pA10273 encodes two proteins that form a heterodimer. SEQ ID NO: 121 and SEQ ID NO: 128 are the monomer sequences.

The term “non-UDP-sugar sugar donor” is used to refer to any glycoside, disaccharide, oligosaccharide, or polysaccharide that contains at least one glucose unit. In one embodiment, the non-UDP-sugar sugar donor is a non-nucleotide-sugar sugar donor.

In one embodiment, the non-UDP-sugar sugar donor is alpha-glucose-1-phosphate. Preferably, alpha-glucose-1-phosphate is generated in situ from an oligo- or poly-saccharide such as starch, sucrose, maltose, cellobiose or gentiobiose by an oligo- or poly-saccharide kinase. In another embodiment, the non-UDP-sugar sugar donor is maltose, cellobiose, beta-glucose-1-phosphate, sucrose, gentiobiose, trehalose, kojibiose, nigerose, isomaltose, beta-beta-trehalose, alpha-beta-trehalose, sophorose, laminaribiose, turanose, maltulose, palatinose, gentiobiulose, another disaccharide, nigerotriose, maltotriose, melezitose, maltotriulose, kestose, starch, cellulose, glycogen, amylose, amylopectin, dextran, dextrin, maltodextrin, glucose syrup, cellodextrin, cyclodextrin, or a steviol glycoside. In some embodiments, the non-UDP-sugar sugar donor is not alpha-glucose-1-phosphate. In some embodiments, the non-UDP-sugar sugar donor is not beta-glucose-1-phosphate.

Optionally, the method of the present disclosure further comprises separating the target steviol glycoside from the target composition. The target steviol glycoside can be separated by any suitable method, such as, for example, crystallization, separation by membranes, centrifugation, extraction, chromatographic separation or a combination of such methods.

In one embodiment, separation produces a composition comprising greater than about 80% by weight of the target steviol glycoside on an anhydrous basis, i.e., a highly purified steviol glycoside composition. In another embodiment, separation produces a composition comprising greater than about 0.5%, about 1%, about 2%, about 3%, about 4%, about 5%, about 10%, about 20%, about 30%, about 40%, about 50%, about 60%, about 70%, about 80%, about 85%, about 90%, about 91%, about 92%, about 93%, about 94%, about 95%, about 96%, about 97%, about 98%, about 99%, about 99.6% by weight of the target steviol glycoside. In particular embodiments, the composition comprises greater than about 95% by weight of the target steviol glycoside.

In another embodiment, contacting the glucosyltransferase polypeptide with a stevia plant extract produces a composition containing multiple steviosides, including Reb A, Reb D Reb M, Reb A isomers, RebD isomer, and Reb M isomers, but enriched in Reb D, Reb M, Reb D isomers and Reb M isomers to more than 1%, ideally more than 2%, 3%, 4%, 5%, 6%, 7%, 8%, 9%, or more than 10%.

The target steviol glycoside can be in any polymorphic or amorphous form, including hydrates, solvates, anhydrous or combinations thereof.

Purified target steviol glycosides can be used in consumable products as a sweetener. Suitable consumer products include, but are not limited to, food, beverages, pharmaceutical compositions, tobacco products, nutraceutical compositions, oral hygiene compositions, and cosmetic compositions.

The present disclosure provides a biocatalytic process for the preparation of a composition comprising a target steviol glycoside from a starting composition comprising a substrate steviol glycoside, wherein the target steviol glycoside comprises one or more additional glucose units than the substrate steviol glycoside.

One object of the disclosure is to provide an efficient biocatalytic method for preparing steviol glycosides, particularly reb D, reb D isomers, reb M and reb M isomers, and rebMp1 isomers from other steviol glycosides and/or mixtures thereof.

As used herein, “biocatalysis” or “biocatalytic” refers to the use of natural catalysts, such as protein enzymes, to perform chemical transformations on organic compounds. Biocatalysis is alternatively known as biotransformation or biosynthesis. Both isolated and whole-cell biocatalysis methods are known in the art. Biocatalyst protein enzymes can be naturally occurring or recombinant proteins.

As used herein, the term “steviol glycoside(s)” refers to a glycoside of steviol, including, but not limited to, naturally occurring steviol glycosides, e.g. steviol-13-O-glucoside, steviol-19-O-glucoside, rubusoside, steviol-1,2-bioside, steviol-1,3-bioside, rubusoside, dulcoside B, dulcoside A, rebaudioside B, rebaudioside G, stevioside, rebaudioside C, rebaudioside F, rebaudioside A, rebaudioside I, rebaudioside E, rebaudioside H, rebaudioside L, rebaudioside K, rebaudioside J, rebaudioside M, rebaudioside D, rebaudioside N, rebaudioside O, rebaudioside Q, synthetic steviol glycosides, e.g. enzymatically glucosylated steviol glycosides and combinations thereof.

### Substrate Steviol Glycoside and Starting Composition

As used herein, “starting composition” refers to any composition (generally an aqueous solution) containing one or more steviol glycosides, where the one or more steviol glycosides serve as the substrate for the biotransformation.

In one embodiment, the starting composition comprises one or more substrate steviol glycosides selected from the group consisting of steviol, steviol-13-O-glucoside, steviol-19-O-glucoside, rubusoside, steviol-1,2-bioside, steviol-1,3-bioside, rubusoside, dulcoside B, dulcoside A, rebaudioside B, rebaudioside G, stevioside, rebaudioside C, rebaudioside F, rebaudioside A, rebaudioside I, rebaudioside E, rebaudioside H, rebaudioside L, rebaudioside K, rebaudioside J, rebaudioside M, rebaudioside D, rebaudioside N, rebaudioside O, rebaudioside Q, synthetic steviol glycosides, e.g. enzymatically glucosylated steviol glycosides and combinations thereof.

In one embodiment, the starting composition comprises the substrate steviol glycoside, stevioside. In one embodiment, the starting composition comprises the substrate steviol glycoside, reb A, or isomers thereof. In one embodiment, the starting composition comprises the substrate steviol glycoside, reb D, or isomers thereof.

The starting composition may be synthetic or purified (partially or entirely), commercially available or prepared. One example of a starting composition useful in the method of the present disclosure is an extract obtained from purification of Stevia rebaudiana plant material (e.g. leaves). Another example of a starting composition is a commercially available stevia extract brought into solution with a solvent. Yet another example of a starting composition is a commercially available mixture of steviol glycosides brought into solution with a solvent. Other suitable starting compositions include by-products of processes to isolate and purify steviol glycosides.

In one embodiment, the starting composition comprises a purified substrate steviol glycoside. For example, the starting composition may comprise greater than about 50%, about 60%, about 70%, about 80%, about 85%, about 90%, about 91%, about 92%, about 93%, about 94%, about 95%, about 96%, about 97%, about 98%, about 99%, or about 99.6% of a particular substrate steviol glycoside by weight on an anhydrous basis.

In another embodiment, the starting composition comprises a partially purified substrate steviol glycoside composition. For example, the starting composition contains greater than about 0.5%, about 1%, about 2%, about 3%, about 4%, about 5%, about 10%, about 20%, about 30%, about 40%, or about 50%, of a particular substrate steviol glycoside by weight on an anhydrous basis.

In another embodiment, the starting composition comprises purified rebaudioside A, or isomers thereof. In a particular embodiment, the starting composition contains greater than about 99% rebaudioside A, or isomers thereof, by weight on an anhydrous basis. In another embodiment, the starting composition comprises partially purified rebaudioside A. In a particular embodiment, the starting composition contains greater than about 50%, about 60%, about 70%, about 80% or about 90% rebaudioside A by weight on an anhydrous basis.

In yet another embodiment, the starting composition comprises purified rebaudioside D, or isomers thereof. In a particular embodiment, the starting composition contains greater than about 99% rebaudioside D, or isomers thereof, by weight on an anhydrous basis. In another embodiment, the starting composition comprises partially purified rebaudioside D. In a particular embodiment, the starting composition contains greater than about 50%, about 60%, about 70%, about 80% or about 90% rebaudioside D by weight on an anhydrous basis.

The steviol glycoside component(s) of the starting composition serve as a substrate(s) for the production of the target steviol glycoside(s), as described herein. The target steviol glycoside target(s) differs chemically from its corresponding substrate steviol glycoside(s) by the addition of one or more glucose units.

### Intermediate Target Steviol Glycoside, Target Steviol Glycoside, and Target Composition

The intermediate target steviol glycoside and/or target steviol glycoside of the present method can be any steviol glycoside that can be prepared by the methods disclosed herein. In one embodiment, the intermediate target steviol glycoside and/or target steviol glycoside is selected from the group consisting of steviol-13-O-glucoside, steviol-19-O-glucoside, rubusoside, steviol-1,2-bioside, steviol-1,3-bioside, rubusoside, dulcoside B, dulcoside A, rebaudioside B, rebaudioside G, stevioside, rebaudioside C, rebaudioside F, rebaudioside A, rebaudioside I, rebaudioside E, rebaudioside H, rebaudioside L, rebaudioside K, rebaudioside J, rebaudioside M, rebaudioside D, rebaudioside N, rebaudioside O, rebaudioside Q, isomers of these steviol glycosides or rebaudioside Mp1 isomers.

In one embodiment, the intermediate target steviol glycoside and/or target steviol glycoside is rebaudioside D (reb D). In another embodiment, the intermediate target steviol glycoside and/or target steviol glycoside is a reb D isomer. In yet another embodiment, the intermediate target steviol glycoside and/or the target steviol glycoside is rebaudioside M (rebM). In still another embodiment, the intermediate target steviol glycoside and/or the target steviol glycoside is a reb M isomer. In still another embodiment, the intermediate target steviol glycoside and/or the target steviol glycoside is a rebMp1 isomer.

In another embodiment, the intermediate target steviol glycoside and/or target steviol glycoside is rebaudioside D, or isomers thereof. In still another embodiment, the intermediate target steviol glycoside and/or the target steviol glycoside is rebaudioside M, or isomers thereof. In still another embodiment, the intermediate target steviol glycoside and/or the target steviol glycoside is a rebaudioside Mp1 isomer. In yet another embodiment, the intermediate target steviol glycoside and/or the target steviol glycoside is rebaudioside D_0.77. In yet another embodiment, the intermediate target steviol glycoside and/or the target steviol glycoside is rebaudioside D_1.07. In yet another embodiment, the intermediate target steviol glycoside and/or the target steviol glycoside is rebaudioside D_1.21. In yet another embodiment, the intermediate target steviol glycoside and/or the target steviol glycoside is rebaudioside D_1.25. In yet another embodiment, the intermediate target steviol glycoside and/or the target steviol glycoside is rebaudioside D_1.29.

In yet another embodiment, the intermediate target steviol glycoside and/or the target steviol glycoside is rebaudioside M_0.66. In yet another embodiment, the target steviol glycoside is rebaudioside M_0.80. In yet another embodiment, the intermediate target steviol glycoside and/or the target steviol glycoside is rebaudioside M_0.92. In yet another embodiment, the intermediate target steviol glycoside and/or the target steviol glycoside is rebaudioside M_1.06. In yet another embodiment, the intermediate target steviol glycoside and/or the target steviol glycoside is rebaudioside M_1.11. In yet another embodiment, the intermediate target steviol glycoside and/or the target steviol glycoside is rebaudioside M_1.17. In yet another embodiment, the intermediate target steviol glycoside and/or the target steviol glycoside is rebaudioside M_1.51.

In yet another embodiment, the intermediate target steviol glycoside and/or the target steviol glycoside is rebaudioside Mp1_0.62. In yet another embodiment, the intermediate target steviol glycoside and/or the target steviol glycoside is rebaudioside Mp1_0.76. In yet another embodiment, the intermediate target steviol glycoside and/or the target steviol glycoside is rebaudioside Mp1_0.82. In yet another embodiment, the intermediate target steviol glycoside and/or the target steviol glycoside is rebaudioside Mp1_0.88. In yet another embodiment, the intermediate target steviol glycoside and/or the target steviol glycoside is rebaudioside Mp1_0.94. In yet another embodiment, the intermediate target steviol glycoside and/or the target steviol glycoside is rebaudioside Mp1_1.09. In yet another embodiment, the intermediate target steviol glycoside and/or the target steviol glycoside is rebaudioside Mp1_1.14. In yet another embodiment, the intermediate target steviol glycoside and/or the target steviol glycoside is rebaudioside Mp1_1.19. In yet another embodiment, the intermediate target steviol glycoside and/or the target steviol glycoside is rebaudioside Mp1_1.27. In yet another embodiment, the intermediate target steviol glycoside and/or the target steviol glycoside is rebaudioside Mp1_1.42. In yet another embodiment, the intermediate target steviol glycoside and/or the target steviol glycoside is rebaudioside Mp1_1.66.

The rebaudioside isomers are defined and identified according to their relative retention times compared to a rebaudioside standard. The relative retention time for rebaudioside D isomers and rebaudioside Mp1 isomers are calculated with respect to the measured rebaudioside D retention time. The relative retention time for rebaudioside M isomers are calculated with respect to the measured rebaudioside M retention time. To account for experimental noise, rebaudioside isomers are classified as a specific isomer if the relative retention time falls within a specified range of the target relative retention time.

Rebaudioside isomers were classified by calculating relative retention times for all rebaudioside isomers detected by the method described in Example 4 (the 4-minute method) and the method described in Example 16 (the 20-minute method). Due to differences in the chromatography gradient between the 4-minute and 20-minute method, the relative retention times for the same isomer could differ between the two methods. Therefore, for some isomers, slightly different relative retention time ranges were used to classify a rebaudioside isomer measured by the 4-minute method versus the 20-minute method.

Rebaudioside D_0.77 is defined as an isomer of rebaudioside D with a relative retention time greater than 0.75 and less than 0.95 compared to the rebaudioside D standard when measured with the 4-minute method or 20-minute method. Rebaudioside D_1.07 is defined as an isomer of rebaudioside D with a relative retention time greater than 1.05 and less than 1.15 compared to the rebaudioside D standard when measured with the 4-minute method or 20-minute method. Rebaudioside D_1.21 is defined as an isomer of rebaudioside D with a relative retention time greater than 1.20 and less than 1.24 compared to the rebaudioside D standard when measured with the 4-minute method and a relative retention time greater than 1.3 and less than 1.4 when measured with the 20-minute method. Rebaudioside D_1.25 is defined as an isomer of rebaudioside D with a relative retention time greater than 1.24 and less than 1.26 compared to the rebaudioside D standard when measured with the 4-minute method and a relative retention time greater than 1.4 and less than 1.45 when measured with the 20-minute method. Rebaudioside D_1.29 is defined as an isomer of rebaudioside D with a relative retention time greater than 1.26 and less than 1.32 compared to the rebaudioside D standard when measured with the 4-minute method and a relative retention time greater than 1.45 and less than 1.6 when measured with the 20-minute method.

Rebaudioside M_0.66 is defined as an isomer of rebaudioside M with a relative retention time greater than 0.6 and less than 0.7 relative to the rebaudioside M standard when measured with the 4-minute method or 20-minute method. Rebaudioside M_0.80 is defined as an isomer of rebaudioside M with a relative retention time greater than 0.7 and less than 0.9 relative to the rebaudioside M standard when measured by the 4-minute method and a relative retention time greater than 0.7 and less than 0.82 when measured with the 20-minute method. Rebaudioside M_0.80 is defined as an isomer of rebaudioside M with a relative retention time greater than 0.7 and less than 0.9 relative to the rebaudioside M standard when measured by the 4-minute method and a relative retention time greater than 0.7 and less than 0.82 when measured with the 20-minute method. Rebaudioside M_0.92 is defined as an isomer of rebaudioside M with a relative retention time greater than 0.9 and less than 0.98 relative to the rebaudioside M standard when measured by the 4-minute method and a relative retention time greater than 0.82 and less than 0.98 when measured with the 20-minute method. Rebaudioside M_1.06 is defined as an isomer of rebaudioside M with a relative retention time greater than 1.04 and less than 1.08 relative to the rebaudioside M standard when measured by the 4-minute method and a relative retention time greater than 1.04 and less than 1.15 when measured with the 20-minute method. Rebaudioside M_1.11 is defined as an isomer of rebaudioside M with a relative retention time greater than 1.10 and less than 1.13 relative to the rebaudioside M standard when measured by the 4-minute method and a relative retention time greater than 1.15 and less than 1.25 when measured with the 20-minute method. Rebaudioside M_1.17 is defined as an isomer of rebaudioside M with a relative retention time greater than 1.15 and less than 1.2 relative to the rebaudioside M standard when measured by the 4-minute method and a relative retention time greater than 1.25 and less than 1.35 when measured with the 20-minute method. Rebaudioside M_1.51 is defined as an isomer of rebaudioside M with a relative retention time greater than 1.4 and less than 1.6 relative to the rebaudioside M standard when measured with the 4-minute method or 20-minute method.

Rebaudioside Mp1_0.62 is defined as a rebaudioside with seven glucose units (i.e. a rebaudioside made by adding a glucose unit to rebaudioside M or a rebaudioside M isomer) with a relative retention time greater than 0.59 and less than 0.68 when measured with the 4-minute method or 20-minute method. Rebaudioside Mp1_0.76 is defined as a rebaudioside with seven glucose units with a relative retention time greater than 0.68 and less than 0.79 when measured with the 4-minute method or 20-minute method. Rebaudioside Mp1_0.82 is defined as a rebaudioside with seven glucose units with a relative retention time greater than 0.79 and less than 0.87 when measured with the 4-minute method or 20-minute method. Rebaudioside Mp1_0.88 is defined as a rebaudioside with seven glucose units with a relative retention time greater than 0.88 and less than 0.91 when measured with the 4-minute method or 20-minute method. Rebaudioside Mp1_0.94 is defined as a rebaudioside with seven glucose units with a relative retention time greater than 0.91 and less than 0.98 when measured with the 4-minute method or 20-minute method. Rebaudioside Mp1_1.09 is defined as a rebaudioside with seven glucose units with a relative retention time greater than 1.04 and less than 1.11 when measured with the 4-minute method or 20-minute method. Rebaudioside Mp1_1.14 is defined as a rebaudioside with seven glucose units with a relative retention time greater than 1.11 and less than 1.17 when measured with the 4-minute method or 20-minute method. Rebaudioside Mp1_1.19 is defined as a rebaudioside with seven glucose units with a relative retention time greater than 1.17 and less than 1.24 when measured with the 4-minute method or 20-minute method. Rebaudioside Mp1_1.27 is defined as a rebaudioside with seven glucose units with a relative retention time greater than 1.24 and less than 1.34 when measured with the 4-minute method or 20-minute method. Rebaudioside Mp1_1.42 is defined as a rebaudioside with seven glucose units with a relative retention time greater than 1.34 and less than 1.45 when measured with the 4-minute method or 20-minute method. Rebaudioside Mp1_1.66 is defined as a rebaudioside with seven glucose units with a relative retention time greater than 1.43 and less than 1.82 when measured with the 4-minute method or 20-minute method.

The target steviol glycoside can be in any polymorphic or amorphous form, including hydrates, solvates, anhydrous or combinations thereof.

In one embodiment, the present disclosure is a biocatalytic process for the production of reb D, or isomers thereof, from reb A, or isomers thereof, where the starting composition comprises the substrate steviol glycoside, reb A. In a particular embodiment, the present disclosure is a biocatalytic process for the production of reb D, or isomers thereof, from reb A, where the starting composition comprises partially purified reb A. In another particular embodiment, the present disclosure is a biocatalytic process for the production of reb D, or isomers thereof, from reb A, where the starting composition comprises purified reb A.

In another embodiment, the present disclosure is a biocatalytic process for the production of a reb D isomer from reb A, where the starting composition comprises the substrate steviol glycoside, reb A. In a particular embodiment, the present disclosure is a biocatalytic process for the production of a reb D isomer from reb A, where the starting composition comprises partially purified reb A. In another particular embodiment, the present disclosure is a biocatalytic process for the production of a reb D isomer from reb A, where the starting composition comprises purified reb A.

In yet another embodiment, the present disclosure is a biocatalytic process for the production of reb M from reb A, where the starting composition comprises the substrate steviol glycoside, reb A. In a particular embodiment, the present disclosure is a biocatalytic process for the production of reb M from reb A, where the starting composition comprises partially purified reb A. In another particular embodiment, the present disclosure is a biocatalytic process for the production of reb M from reb A, where the starting composition comprises purified reb A.

In still another embodiment, the present disclosure is a biocatalytic process for the production of a reb M isomer from reb A, where the starting composition comprises the substrate steviol glycoside, reb A. In a particular embodiment, the present disclosure is a biocatalytic process for the production of a reb M isomer from reb A, where the starting composition comprises partially purified reb A. In another particular embodiment, the present disclosure is a biocatalytic process for the production of a reb M isomer from reb A, where the starting composition comprises purified reb A.

In still another embodiment, the present disclosure is a biocatalytic process for the production of a reb Mp1 isomer from reb A, where the starting composition comprises the substrate steviol glycoside, reb A. In a particular embodiment, the present disclosure is a biocatalytic process for the production of a reb Mp1 isomer from reb A, where the starting composition comprises partially purified reb A. In another particular embodiment, the present disclosure is a biocatalytic process for the production of a reb Mp1 isomer from reb A, where the starting composition comprises purified reb A.

In yet another embodiment, the present disclosure is a biocatalytic process for the production of reb M from reb D, where the starting composition comprises the substrate steviol glycoside, reb D. In a particular embodiment, the present disclosure is a biocatalytic process for the production of reb M from reb D, where the starting composition comprises partially purified reb D. In another particular embodiment, the present disclosure is a biocatalytic process for the production of reb M from reb D, where the starting composition comprises purified reb D.

In still another embodiment, the present disclosure is a biocatalytic process for the production of a reb M isomer from reb D, where the starting composition comprises the substrate steviol glycoside, reb D. In a particular embodiment, the present disclosure is a biocatalytic process for the production of a reb M isomer from reb D, where the starting composition comprises partially purified reb D. In another particular embodiment, the present disclosure is a biocatalytic process for the production of a reb M isomer from reb D, where the starting composition comprises purified reb D.

In still another embodiment, the present disclosure is a biocatalytic process for the production of a reb Mp1 isomer from reb D, where the starting composition comprises the substrate steviol glycoside, reb D. In a particular embodiment, the present disclosure is a biocatalytic process for the production of a reb Mp1 isomer from reb D, where the starting composition comprises partially purified reb D. In another particular embodiment, the present disclosure is a biocatalytic process for the production of a reb Mp1 isomer from reb D, where the starting composition comprises purified reb D.

In still another embodiment, the present disclosure is a biocatalytic process for the production of reb M, a rebM isomer, or a rebMp1 isomer from a reb D isomer, where the starting composition comprises a reb D isomer. In a particular embodiment, the present disclosure is a biocatalytic process for the production of reb M, a reb M isomer or a rebMp1 isomer from a reb D isomer, where the starting composition comprises the partially purified reb D isomer. In another particular embodiment, the present disclosure is a biocatalytic process for the production of reb M, a reb M isomer or a rebMp1 isomer from a reb D isomer, where the starting composition comprises the purified reb D isomer.

Optionally, the method of the present disclosure further comprises separating the intermediate target steviol glycoside from the intermediate composition. Optionally, the method of the present disclosure further comprises separating the target steviol glycoside from the target composition. The target steviol glycoside can be separated by any suitable method, such as, for example, crystallization, separation by membranes, centrifugation, extraction, chromatographic separation or a combination of such methods.

In particular embodiments, the methods described herein results in a partially purified intermediate target steviol glycoside and/or target steviol glycoside composition. The term “partially purified”, as used herein, refers to a composition having greater than about 0.5% by weight of the target steviol glycoside on an anhydrous basis. In one embodiment, the partially purified intermediate target steviol glycoside and/or target steviol glycoside composition contains greater than about 90% by weight of the target steviol glycoside on an anhydrous basis, such as, for example, greater than about 0.5%, about 1%, about 2%, about 3%, about 4%, about 5%, about 10%, about 20%, about 30%, about 40%, or about 50% target steviol glycoside content on an anhydrous basis.

In particular embodiments, the methods described herein results in a purified target intermediate target steviol glycoside and/or steviol glycoside composition. The term “purified”, as used herein, refers to a composition having greater than about 50% by weight of the target steviol glycoside on an anhydrous basis. In one embodiment, the partially purified target steviol glycoside composition contains greater than about 50%, about 60%, about 70%, about 80%, about 85%, or about 90% target steviol glycoside content on an anhydrous basis.

In particular embodiments, the methods described herein results in a highly purified intermediate target steviol glycoside and/or target steviol glycoside composition. The term “highly purified”, as used herein, refers to a composition having greater than about 80% by weight of the target steviol glycoside on an anhydrous basis. In one embodiment, the highly purified target steviol glycoside composition contains greater than about 90% by weight of the target steviol glycoside on an anhydrous basis, such as, for example, 91% greater than about 92%, greater than about 93%, greater than about 94%, greater than about 95%, greater than about 95%, greater than about 97%, greater than about 98%, greater than about 99%, or greater than about 99.6% target steviol glycoside content on an anhydrous basis.

In one embodiment, the biocatalytic methods of the present disclosure is carried out more than one time, such that the target steviol glycoside produced by a first biocatalytic process serves as the substrate steviol glycoside (which could also be considered an intermediate target steviol glycoside) for a second biocatalytic process in which the target steviol glycoside is produced.

In a particular embodiment, the present disclosure provides a biocatalytic process for preparing a composition comprising a target steviol glycoside by contacting a starting composition comprising a substrate steviol glycoside with a first glucosyltransferase polypeptide that utilizes a non-UDP-sugar sugar donor, thereby producing a composition comprising an intermediate target steviol glycoside comprising one or more additional glucose units than the substrate steviol glycoside; contacting the intermediate composition comprising the intermediate target steviol glycoside with a second glucosyltransferase polypeptide, thereby producing a target steviol glycoside comprising one or more additional glucose units than the intermediate target steviol glycoside. Depending on the number of times the method is carried out, there may be one or more intermediate target steviol glycosides (e.g., a first intermediate target steviol glycoside, a second intermediate target steviol glycoside, a third intermediate target steviol glycoside) involved in the production of the target steviol glycoside.

### Glucotransferase Polypeptides

The present methods are biocatalytic, i.e., utilizes a biological catalyst. In one embodiment, the biocatalyst is protein enzyme. In a particular embodiment, the biocatalyst is a glucosyltransferase polypeptide that utilizes non-UDP-sugar sugar donors. The glucosyltransferase polypeptides can be any glucosyltransferase polypeptides capable of adding at least one glucose unit to the substrate steviol glycoside to provide the intermediate target steviol glycoside and/or the target steviol glycoside.

The present disclosure also provides a recombinant glycosyltransferase polypeptide comprising an amino acid sequence that is at least 60%, at least 65%, at least 70%, at least 75%, at least 80%, at least 85%, at least 90%, at least 95%, at least 98%, or at least 99% identical to an amino acid sequence selected from the group consisting of SEQ ID NOs: 1-128. The recombinant glycosyltransferase polypeptides of the present disclosure may comprise, or consist of, an amino acid sequence that is at least 60% identical to an amino acid sequence selected from the group consisting of SEQ ID NOs: 2, 3, 22, 29, 39, 41, 42, 45, 49, 50, 51, 52, 53, 54, 55, 56, 62, 63, 72, 87, 90, 91, 94, 97, 98, 99, 100, 101, 102, 109, 110, 111, 112, 113, 115, 117, 118, 119, 121, 123, 124, 125, 126, and 128. In some embodiments, the recombinant glycosyltransferase polypeptide comprises, or consists of, an amino acid sequence that is at least 60% identical to an amino acid sequence selected from the group consisting of SEQ ID NOs: 1, 19, 29, 32, 41, 42, 43, 44, 45, 46, 47, 48, 49, 50, 51, 52, 53, 54, 55, 56, 58, 61, 65, 67, 68, 81, 87, 90, 91, 92, 93, 94, 95, 96, 97, 98, 99, 100, 101, 102, 105, 106, 116, 120, 122, and 127.

In some embodiments, the glycosyltransferase polypeptide comprises, or consists of, an amino acid sequence selected from the group consisting of SEQ ID NOs: 1-128. In other embodiments, the glycosyltransferase polypeptide comprises, or consists of, an amino acid sequence selected from the group consisting of SEQ ID NOs: 1, 19, 29, 32, 41, 42, 43, 44, 45, 46, 47, 48, 49, 50, 51, 52, 53, 54, 55, 56, 58, 61, 65, 67, 68, 81, 87, 90, 91, 92, 93, 94, 95, 96, 97, 98, 99, 100, 101, 102, 105, 106, 116, 120, 122, and 127. In other embodiments, the glycosyl-transferase polypeptide comprises, or consists of, an amino acid sequence selected from the group consisting of SEQ ID NOs: 2, 3, 22, 29, 39, 41, 42, 45, 49, 50, 51, 52, 53, 54, 55, 56, 62, 63, 72, 87, 90, 91, 94, 97, 98, 99, 100, 101, 102, 109, 110, 111, 112, 113, 115, 117, 118, 119, 121, 123, 124, 125, 126, and 128. In further embodiments, the glycosyltransferase polypeptide is capable of transferring a sugar moiety to a substrate steviol glycoside.

In one embodiment, the glucosyltransferase polypeptide is produced in a host, such as a microorganism. For example, a DNA sequence encoding glucosyltransferase is cloned into an expression vector and transferred into a production host such as a microbe, e.g., a bacteria. Non-limiting examples of suitable hosts include E. coli, Saccharomyces sp., Aspergillus sp., Pichia sp., Bacillus sp. The overexpressed protein can be isolated from the cell extract based on its physical and chemical properties, using techniques known in the art. Representative non-limiting techniques for isolating glucosyltransferase from a host include centrifugation, electrophoresis, liquid chromatography, ion exchange chromatography, gel filtration chromatography or affinity chromatography.

Glucosyltransferase polypeptide can be provided as a crude, semi-purified and purified enzyme preparation(s).

In one embodiment, the glucosyltransferase polypeptide is free. In another embodiment, the glucosyltransferase polypeptide is immobilized. For example, glucosyltransferase may be immobilized to a solid support made from inorganic or organic materials. Non-limiting examples of solid supports suitable to immobilize glucosyltransferase polypeptide include derivatized cellulose or glass, ceramics, methacrylate, styrene, acrylic, metal oxides or membranes. Glucosyltransferase may be immobilized to the solid support, for example, by covalent attachment, adsorption, cross-linking, entrapment or encapsulation.

The reaction medium for conversion is generally aqueous, e.g., purified water, buffer or a combination thereof In a particular embodiment, the reaction medium is a buffer. Suitable buffers include, but are not limited to, acetate buffer, citrate buffer HEPES, and phosphate buffer. In a particular embodiment, the reaction medium is acetate buffer. In another particular embodiment, the reaction medium is HEPES buffer. The reaction medium can also be, alternatively, an organic solvent.

In one embodiment, the glucosyltransferase is provided in the form of a whole cell system, such as a living or non-living microbial cell. The whole cell system may optionally be immobilized, as well, utilizing the techniques identified above with respect to immobilization of the enzyme.

In one embodiment, the glucosyltransferase is any glucosyltransferase capable of adding at least one glucose unit to reb A using a non-UDP-sugar sugar donor, thereby producing stevioside. The glucosyltransferase may be, for example, one of SEQ ID NOs: 1-128.

In another embodiment, the glucosyltransferase polypeptide is any glucosyltransferase capable of adding at least one glucose unit to rebD or a rebD isomer using a non-UDP-sugar sugar donor, thereby producing reb M or a reb M isomer. The glucosyltransferase polypeptide may be, for example, one of SEQ ID NOs: 2, 3, 22, 29, 39, 41, 42, 45, 49, 50, 51, 52, 53, 54, 55, 56, 62, 63, 72, 87, 90, 91, 94, 97, 98, 99, 100, 101, 102, 109, 110, 111, 112, 113, 115, 117, 118, 119, 121, 123, 124, 125, 126, and 128.

### The Conversion of Reb A to Reb D and/or Reb D Isomers

In one embodiment, a starting composition comprising reb A is contacted with a glucosyltransferase capable of catalyzing the reaction of a sugar donor (alpha-glucose-1-phosphate, cellobiose, sucrose, maltose, or gentiobiose) and reb A to produce reb D and/or reb D isomers. In one embodiment, the starting composition comprises partially purified reb A. In another embodiment, the starting composition comprises purified reb A. In a particular embodiment, the starting composition comprises >99% reb A. In a particular embodiment, the starting composition comprises greater than 0.5%, about 1%, about 2%, about 3%, about 4%, about 5%, about 10%, about 15%, about 20%, about 30%, about 40%, about 50%, about 60%, about 70% about 80%, about 90%, about 91%, about 92%, about 93%, about 94%, about 95%, about 96%, about 97%, about 98%, about 99%, or about 99.6% reb A.

In a particular embodiment, the glucosyltransferase polypeptide comprises or consists of an amino acid sequence that is at least 60%, at least 65%, at least 70%, at least 75%, at least 80%, at least 85%, at least 90%, at least 95%, at least 98%, at least 99% or 100% identical to an amino acid sequence selected from the group consisting of SEQ ID NOs: 1, 19, 29, 32, 41, 42, 43, 44, 45, 46, 47, 48, 49, 50, 51, 52, 53, 54, 55, 56, 58, 61, 65, 67, 68, 81, 87, 90, 91, 92, 93, 94, 95, 96, 97, 98, 99, 100, 101, 102, 105, 106, 116, 120, 122, and 127.

In some embodiments, the glucosyltransferase polypeptide is prepared by expression in a host microorganism. Suitable host microorganisms include, but are not limited to, E. coli, Saccharomyces sp., Aspergillus sp., Pichia sp., Bacillus sp. In a particular embodiment, the glucosyltransferase is expressed in E. coli.

The glucosyltransferase polypeptide can be provided free or in an immobilized form. The enzyme preparation may be crude, semi-purified and purified. In one embodiment, the glucosyltransferase is provided as a whole-cell system, e.g., a living or non-living microbial cell, or whole microbial cells, cell lysate and/or any other form of known in the art.

The reaction medium for conversion is generally aqueous, and can be purified water, buffer or a combination thereof. In a particular embodiment, the reaction medium is a buffer. Suitable buffers include, but are not limited to, acetate buffer, citrate buffer, phosphate buffer, and HEPES buffer. In one embodiment, the reaction medium is acetate buffer. In another embodiment, the reaction medium is HEPES buffer.

In one embodiment, conversion of reb A to reb D and/or rebD isomers further comprises the addition of compounds other than sugar donor (alpha-glucose1-phosphate, cellobiose, sucrose, maltose, or gentiobiose), reb A and the glucosyltransferase. For example, in some embodiments, the reaction medium includes pyridoxal phosphate (PLP) and/or one or more metal ions. Exemplary metal ions include, but are not limited to Ca²⁺, Mg²⁺, Mn²⁺, Fe²⁺, Fe³⁺, CO²⁺, Co³⁺, Ni²⁺, Cu²⁺, Zn²⁺. For example, the reaction medium includes one or more of (NH₄)MgPO₄, Mg₆Al₂(CO₃)(OH)₁₆, MgBr₂, (MgCO₃)₄.Mg(OH)₂, MgCl₂, MgCrO₄, MgF₂, Mg(IO₃)₂, MgI₂, Mg(NO₃)₂, Mg(ClO₄)₂, (CH₃)₃COMgOC(CH₃)₃, Mg(MnO₄)₂, MgHPO₄, Mg₃(PO₄)₂, MgSO₄, CaBr₂, CaCO₃, CaCl₂, CaNCN, CaF₂, H₂Ca, Ca(OH)₂, Ca(IO₃)₂, CaI₂, Ca(NO₃)₂, Ca(NO₂)₂, CaC₂O₄, Ca(ClO₄)₂, [Ca₅(OH)(PO₄)₃]x, Ca(H₂PO₄)₂, Ca₂P₂O₇, CaSO₄, Ca(SCN)₂, MnBr₂, MnCO₃, MnCl₂, [C₆H₁₁(CH₂)₃CO2]₂Mn, MnF₂, (HCO₂)₂Mn, MnI₂, Mn(NO₃)₂, Mn(ClO₄)₂, and MnSO₄.

The step of contacting the starting composition with the glycosyltransferase polypeptide and the non-UDP-sugar sugar donor can be carried out at temperature between about 0° C. and about 60° C., such as, for example, about 10° C., about 20° C., about 30° C., about 40° C., about 50° C. or about 60° C. In a particular embodiment, the reaction is carried out at about 30° C.

The step of contacting the starting composition with the glycosyltransferase polypeptide and the non-UDP-sugar sugar donor can carried out in a duration of time between 1 hour and 1 week, such as, for example, about 6 hours, about 12 hours, about 24 hours, about 48 hours, about 72 hours, about 120 hours, about 3 days, about 4 days, about 5 days, about 6 days or about 7 days. In a particular embodiment, the reaction is carried out for about 24 hours.

The reaction can be monitored by suitable method including, but not limited to, HPLC, LCMS, TLC, IR or NMR.

In one embodiment, the conversion of reb A to reb D and/or reb D isomer(s) is at least about 2% complete, as determined by any of the methods mentioned above. In a particular embodiment, the conversion of reb A to reb D and/or rebD isomer(s) is at least about 10% complete, at least about 20% complete, at least about 30% complete, at least about 40% complete, at least about 50% complete, at least about 60% complete, at least about 70% complete, at least about 80% complete or at least about 90% complete. In a particular embodiment, the conversion of reb A to reb D and/or rebD isomer(s) is at least about 95% complete. In some embodiments, wherein at least about 5%, about 10%, about 20%, about 30%, about 40%, about 50%, about 60%, about 70%, about 80%, or about 90% of the reb A in the starting composition is converted to reb D and/or rebD isomer(s).

### The Conversion of Reb D to Reb M and/or Reb M Isomers

In one embodiment, a starting composition comprising reb D is contacted with a glucosyltransferase capable of catalyzing the reaction of a sugar donor (alpha-glucose-1-phosphate, cellobiose, sucrose, maltose, or gentiobiose) and reb D to produce reb M and/or reb M isomers. In one embodiment, a reb D isomer can be used in place of rebD as the substrate of the reaction. In one embodiment, the starting composition comprises partially purified reb D. In another embodiment, the starting composition comprises purified reb D. In a particular embodiment, the starting composition comprises >99% reb D. In a particular embodiment, the starting composition comprises greater than 0.5%, about 1%, about 2%, about 3%, about 4%, about 5%, about 10%, about 15%, about 20%, about 30%, about 40%, about 50%, about 60%, about 70% about 80% about 90%, about 91%, about 92%, about 93%, about 94%, about 95%, about 96%, about 97%, about 98%, about 99%, or about 99.6% reb D and/or its isomers.

In a particular embodiment, the glucosyltransferase polypeptide comprising an amino acid sequence that is at least 60%, at least 65%, at least 70%, at least 75%, at least 80%, at least 85%, at least 90%, at least 95%, at least 98%, at least 99% or 100% identical to an amino acid sequence selected from the group consisting of SEQ ID NOs: 2, 3, 22, 29, 39, 41, 42, 45, 49, 50, 51, 52, 53, 54, 55, 56, 62, 63, 72, 87, 90, 91, 94, 97, 98, 99, 100, 101, 102, 109, 110, 111, 112, 113, 115, 117, 118, 119, 121, 123, 124, 125, 126, and 128.

In some embodiments, the glucosyltransferase polypeptide is prepared by expression in a host microorganism. Suitable host microorganisms include, but are not limited to, E. coli, Saccharomyces sp., Aspergillus sp., Pichia sp., Bacillus sp. In a particular embodiment, the glucosyltransferase is expressed in E. coli.

The glucosyltransferase polypeptide can be provided free or in an immobilized form. The enzyme preparation may be crude, semi-purified and purified. In one embodiment, the glucosyltransferase is provided as a whole-cell system, e.g., a living or non-living microbial cell, or whole microbial cells, cell lysate and/or any other form of known in the art.

The reaction medium for conversion is generally aqueous, and can be purified water, buffer or a combination thereof. In a particular embodiment, the reaction medium is a buffer. Suitable buffers include, but are not limited to, acetate buffer, citrate buffer, phosphate buffer, and HEPES buffer. In one embodiment, the reaction medium is acetate buffer. In another embodiment, the reaction medium is HEPES buffer.

In one embodiment, conversion of reb D to reb M or rebM isomers further comprises the addition of compounds other than sugar donor (alpha-glucose-1-phosphate, cellobiose, sucrose, maltose, or gentiobiose), reb D and the glucosyltransferase. For example, in some embodiments, the reaction medium includes pyridoxal phosphate (PLP) and/or one or more metal ions. Exemplary metal ions includes, but are not limited to Ca²⁺, Mg²⁺, Mn²⁺, Fe²⁺, Fe³⁺, CO²⁺, Co³⁺, Ni²⁺, Cu²⁺, Zn²⁺. For example, the reaction medium includes one or more of (NH₄)MgPO₄, Mg₆Al₂(CO₃)(OH)₁₆, MgBr₂, (MgCO₃)₄.Mg(OH)₂, MgCl₂, MgCrO₄, MgF₂, Mg(IO₃)₂, MgI₂, Mg(NO₃)₂, Mg(ClO₄)₂, (CH₃)₃COMgOC(CH₃)₃, Mg(MnO₄)₂, MgHPO₄, Mg₃(PO₄)₂, MgSO₄, CaBr₂, CaCO₃, CaCl₂, CaNCN, CaF₂, H₂Ca, Ca(OH)₂, Ca(IO₃)₂, CaI₂, Ca(NO₃)₂, Ca(NO₂)₂, CaC₂O₄, Ca(ClO₄)₂, [Ca₅(OH)(PO₄)₃]x, Ca(H₂PO₄)₂, Ca₂P₂O₇, CaSO₄, Ca(SCN)₂, MnBr₂, MnCO₃, MnCl₂, [C₆H₁₁(CH₂)₃CO2]₂Mn, MnF₂, (HCO₂)₂Mn, MnI₂, Mn(NO₃)₂, Mn(ClO₄)₂, and MnSO₄.

The step of contacting the starting composition with the glycosyltransferase polypeptide and the non-UDP-sugar sugar donor can be carried out at temperature between about 0° C. and about 60° C., such as, for example, about 10° C., about 20° C., about 30° C., about 40° C., about 50° C. or about 60° C. In a particular embodiment, the reaction is carried out at about 30° C.

The step of contacting the starting composition with the glycosyltransferase polypeptide and the non-UDP-sugar sugar donor can be carried out in a duration of time between 1 hour and 1 week, such as, for example, about 6 hours, about 12 hours, about 24 hours, about 48 hours, about 72 hours, about 120 hours, about 3 days, about 4 days, about 5 days, about 6 days or about 7 days. In a particular embodiment, the reaction is carried out for about 24 hours.

The reaction can be monitored by suitable method including, but not limited to, HPLC, LCMS, TLC, IR or NMR.

In one embodiment, the conversion of reb D to reb M and/or reb M isomer(s) is at least about 2% complete, as determined by any of the methods mentioned above. In a particular embodiment, the conversion of reb D to reb M and/or rebM isomer(s) is at least about 10% complete, at least about 20% complete, at least about 30% complete, at least about 40% complete, at least about 50% complete, at least about 60% complete, at least about 70% complete, at least about 80% complete or at least about 90% complete. In a particular embodiment, the conversion of rebD to rebM and/or rebM isomer(s) is at least about 95% complete. In some embodiments, wherein at least about 5%, about 10%, about 20%, about 30%, about 40%, about 50%, about 60%, about 70%, about 80%, or about 90% of the rebD in the starting composition is converted to rebM and/or rebM isomer(s).

### Methods of Transferring a Sugar Moiety to a Substrate Steviol Glycoside

In embodiments, provided herein are methods for transferring a sugar moiety to a substrate steviol glycoside. The methods comprises contacting the substrate steviol glycoside with a glycosyltransferase polypeptide and a non-UDP-sugar sugar donor. In some embodiments, the glycosyltransferase polypeptide comprises an amino acid sequence that is at least 60%, at least 65%, at least 70%, at least 75%, at least 80%, at least 85%, at least 90%, at least 95%, at least 98%, or at least 99% identical to an amino acid sequence selected from the group consisting of SEQ ID NOs: 1-128. In other embodiments, the glycosyltransferase polypeptide comprises an amino acid sequence selected from the group consisting of SEQ ID NOs: 1-128.

The recombinant glycosyltransferase polypeptide of the present invention teaches that the glycosyltransferase polypeptide comprises an amino acid sequence that is at least 60%, at least 65%, at least 70%, at least 75%, at least 80%, at least 85%, at least 90%, at least 95%, at least 98%, or at least 99% identical to an amino acid sequence selected from the group consisting of SEQ ID NOs: 2, 3, 22, 29, 39, 41, 42, 45, 49, 50, 51, 52, 53, 54, 55, 56, 62, 63, 72, 87, 90, 91, 94, 97, 98, 99, 100, 101, 102, 109, 110, 111, 112, 113, 115, 117, 118, 119, 121, 123, 124, 125, 126, and 128. In some embodiments, the recombinant glycosyltransferase polypeptide comprises an amino acid sequence that is at least 60%, at least 65%, at least 70%, at least 75%, at least 80%, at least 85%, at least 90%, at least 95%, at least 98%, or at least 99% identical to an amino acid sequence selected from the group consisting of SEQ ID NOs: 1, 19, 29, 32, 41, 42, 43, 44, 45, 46, 47, 48, 49, 50, 51, 52, 53, 54, 55, 56, 58, 61, 65, 67, 68, 81, 87, 90, 91, 92, 93, 94, 95, 96, 97, 98, 99, 100, 101, 102, 105, 106, 116, 120, 122, and 127.

In some embodiments, the substrate steviol glycoside is steviol, steviol-13-O-glucoside, steviol-19-O-glucoside, rubusoside, steviol-1,2-bioside, steviol-1,3-bioside, rubusoside, dulcoside B, dulcoside A, rebaudioside B, rebaudioside G, stevioside, rebaudioside C, rebaudioside F, rebaudioside A, rebaudioside I, rebaudioside E, rebaudioside H, rebaudioside L, rebaudioside K, rebaudioside J, rebaudioside M, rebaudioside D, rebaudioside N, rebaudioside O, rebaudioside Q, an isomer thereof, a synthetic steviol glycoside or combinations thereof. In other embodiments, the substrate steviol glycoside is rebaudioside A, an isomer thereof, or combinations thereof. In further embodiments, the substrate steviol glycoside is rebaudioside D, an isomer thereof, or combinations thereof.

In some embodiments, the non-UDP-sugar sugar donor is alpha-glucose-1-phosphate, beta-glucose-1-phosphate, cellobiose, sucrose, maltose, gentiobiose, trehalose, kojibiose, nigerose, isomaltose, beta-beta-trehalose, alpha-beta-trehalose, sophorose, laminaribiose, turanose, maltulose, palatinose, gentiobiulose, nigerotriose, maltotriose, melezitose, maltotriulose, kestose, starch, cellulose, glycogen, amylose, amylopectin, dextran, dextrin, maltodextrin, glucose syrup, cellodextrin, cyclodextrin, a non-UDP nucleotide sugar (i.e. ADP-glucose, GDP-glucose, CDP-glucose, TDP-glucose), or a steviol glycoside. In other embodiments, the non-UDP-sugar sugar donor is not alpha-glucose-1-phosphate. In further embodiments, the non-UDP-sugar sugar donor is not beta-glucose-1-phosphate.

In some embodiments, the glucosyltransferase polypeptide is expressed in a host microorganism. In some embodiments, the host microorganism is E. coli, Saccharomyces sp., Aspergillus sp., Pichia sp., or Bacillus sp.

In some embodiments, the glucosyltransferase polypeptide is immobilized to a solid support. In some embodiments, the solid support is derivatized cellulose, glass, ceramic, methacrylate, styrene, acrylic, a metal oxide, or a membrane. In other embodiments, the glucosyltransferase polypeptide is immobilized to the solid support by covalent attachment, adsorption, cross-linking, entrapment, or encapsulation.

In some embodiments, the contacting the substrate steviol glycoside with the glycosyltransferase polypeptide and the non-UDP-sugar sugar donor is in a reaction medium comprising pyridoxal phosphate (PLP) and/or one or more metal ions. In some embodiments, the contacting the substrate steviol glycoside with the glycosyltransferase polypeptide and the non-UDP-sugar sugar donor is at temperature between about 0° C. and about 60° C. In other embodiments, the contacting the substrate steviol glycoside with the glycosyltransferase polypeptide and the non-UDP-sugar sugar donor is at temperature about 30° C. In further embodiments, the contacting the substrate steviol glycoside with the glycosyltransferase polypeptide and the non-UDP-sugar sugar donor is carried out in a duration of time between 1 hour and 1 week. In further embodiments, the contacting the substrate steviol glycoside with the glycosyltransferase polypeptide and the non-UDP-sugar sugar donor is carried out in a duration of time of about 24 hours.

### Methods of Producing a Target Steviol Glycoside

In some embodiments, provided herein is a method for producing a target steviol glycoside composition, comprising the steps of: (a) providing a starting composition comprising greater than about 0.5%, about 1%, about 2%, about 3%, about 4%, about 5%, about 10%, about 20%, about 30%, about 40%, about 50%, about 60%, about 70%, about 80%, about 90%, about 92%, about 93%, about 94%, about 95%, about 96%, about 97%, about 98%, about 99%, or about 99.6% of a substrate steviol glycoside by weight on an anhydrous basis; (b) contacting the starting composition with a glycosyltransferase polypeptide and a non-UDP-sugar sugar donor; and (c) producing a target composition comprising a target steviol glycoside.

In some embodiments, the glycosyltransferase polypeptide comprises an amino acid sequence that is at least 60%, at least 65%, at least 70%, at least 75%, at least 80%, at least 85%, at least 90%, at least 95%, at least 98%, or at least 99% identical to an amino acid sequence selected from the group consisting of SEQ ID NOs: 1-128. Combinations may also be used, including, for example, SEQ ID NOS 121 and 128, which form a heterodimer.

In some embodiments, the substrate steviol glycoside is rebaudioside A, or isomers thereof, and the glycosyltransferase polypeptide comprises an amino acid sequence selected from the group consisting of SEQ ID NOs: 1, 19, 29, 32, 41, 42, 43, 44, 45, 46, 47, 48, 49, 50, 51, 52, 53, 54, 55, 56, 58, 61, 65, 67, 68, 81, 87, 90, 91, 92, 93, 94, 95, 96, 97, 98, 99, 100, 101, 102, 105, 106, 116, 120, 122, and 127.

In some embodiments, the substrate steviol glycoside is rebaudioside D, or isomers thereof, and the glycosyltransferase polypeptide comprises an amino acid sequence selected from the group consisting of SEQ ID NOs: 2, 3, 22, 29, 39, 41, 42, 45, 49, 50, 51, 52, 53, 54, 55, 56, 62, 63, 72, 87, 90, 91, 94, 97, 98, 99, 100, 101, 102, 109, 110, 111, 112, 113, 115, 117, 118, 119, 121, 123, 124, 125, 126, and 128.

In some embodiments, the target composition comprises greater than about 0.5%, about 1%, about 2%, about 3%, about 4%, about 5%, about 10%, about 20%, about 30%, about 40%, about 50%, about 60%, about 70%, about 80%, about 85%, about 90%, about 91%, about 92%, about 93%, about 94%, about 95%, about 96%, about 97%, about 98%, about 99%, about 99.6% of the target steviol glycoside by weight on an anhydrous basis.

In some embodiments, the substrate steviol glycoside is steviol, steviol-13-O-glucoside, steviol-19-O-glucoside, rubusoside, steviol-1,2-bioside, steviol-1,3-bioside, rubusoside, dulcoside B, dulcoside A, rebaudioside B, rebaudioside G, stevioside, rebaudioside C, rebaudioside F, rebaudioside A, rebaudioside I, rebaudioside E, rebaudioside H, rebaudioside L, rebaudioside K, rebaudioside J, rebaudioside M, rebaudioside D, rebaudioside N, rebaudioside O, rebaudioside Q, an isomer thereof, a synthetic steviol glycoside or combinations thereof. In other embodiments, the non-UDP-sugar sugar donor is alpha-glucose-1-phosphate, beta-glucose-1-phosphate, cellobiose, sucrose, maltose, gentiobiose, trehalose, kojibiose, nigerose, isomaltose, beta-beta-trehalose, alpha-beta-trehalose, sophorose, laminaribiose, turanose, maltulose, palatinose, gentiobiulose, nigerotriose, maltotriose, melezitose, maltotriulose, kestose, starch, cellulose, glycogen, amylose, amylopectin, dextran, dextrin, maltodextrin, glucose syrup, cellodextrin, or cyclodextrin. In other embodiments, the non-UDP-sugar sugar donor is not alpha-glucose-1-phosphate. In further embodiments, the non-UDP-sugar sugar donor is not beta-glucose-1-phosphate.

In some embodiments, at least about 5%, about 10%, about 20%, about 30%, about 40%, about 50%, about 60%, about 70%, about 80%, or about 90% of the substrate steviol glycoside in the starting composition is converted to the target steviol glycoside.

In some embodiments, the substrate steviol glycoside is rebaudioside A, or isomers thereof and the target steviol glycoside is rebaudioside D, or isomers thereof. In other embodiments, the target steviol glycoside is a rebaudioside D isomer. In further embodiment, the rebaudioside D isomer is rebaudioside D_1.07, rebaudioside D_0.77, rebaudioside D_1.21, rebaudioside D_1.25, or rebaudioside D_1.29.

In some embodiments, the substrate steviol glycoside is rebaudioside A, or isomers thereof; and the target steviol glycoside is rebaudioside M, or isomers thereof. In other embodiments, the target steviol glycoside is a rebaudioside M isomer. In further embodiment, the rebaudioside M isomer is rebaudioside M_0.66, rebaudioside M_0.80, rebaudioside M_0.92, rebaudioside M_1.06, rebaudioside M_1.11, rebaudioside M_1.17, or rebaudioside M_1.51.

In some embodiments, the substrate steviol glycoside is rebaudioside A, or isomers thereof; and the target steviol glycoside is a rebaudioside Mp1 isomer. In further embodiment, the rebaudioside Mp1 isomer is rebaudioside Mp1_0.62, rebaudioside Mp1_0.76, rebaudioside Mp1_0.82, rebaudioside Mp1_0.88, rebaudioside Mp1_0.94, rebaudioside Mp1_1.09, rebaudioside Mp1_1.14, rebaudioside Mp1_1.19, rebaudioside Mp1_1.27, rebaudioside Mp1_1.42, or rebaudioside Mp1_1.66.

In some embodiments, the substrate steviol glycoside is rebaudioside D, or isomers thereof; and the target steviol glycoside is rebaudioside M, or isomers thereof. In other embodiments, the substrate steviol glycoside is a rebaudioside D isomer. In further embodiments, the rebaudioside D isomer is rebaudioside D_1.07, rebaudioside D_0.77, rebaudioside D_1.21, rebaudioside D_1.25, or rebaudioside D_1.29.

In some embodiments, the target steviol glycoside is a rebaudioside M isomer. In other embodiments, the rebaudioside M isomer is rebaudioside M_0.66, rebaudioside M_0.80, rebaudioside M_0.92, rebaudioside M_1.06, rebaudioside M_1.11, rebaudioside M_1.17, or rebaudioside M_1.51.

In some embodiments, the target steviol glycoside is a rebaudioside Mp1 isomer. In other embodiments, the rebaudioside Mp1 isomer is rebaudioside Mp1_0.62, rebaudioside Mp1_0.76, rebaudioside Mp1_0.82, rebaudioside Mp1_0.88, rebaudioside Mp1_0.94, rebaudioside Mp1_1.09, rebaudioside Mp1_1.14, rebaudioside Mp1_1.19, rebaudioside Mp1_1.27, rebaudioside Mp1_1.42, rebaudioside Mp1_1.66.

In some embodiments, the starting composition is a Stevia rebaudiana extract. In other embodiments, the glucosyltransferase polypeptide is expressed in a host microorganism. In further embodiments, the host microorganism is E. coli, Saccharomyces sp., Aspergillus sp., Pichia sp., or Bacillus sp.

In some embodiments, the glucosyltransferase polypeptide is immobilized to a solid support. In other embodiments, the solid support is derivatized cellulose, glass, ceramic, methacrylate, styrene, acrylic, a metal oxide, or a membrane.

In some embodiments, the step of contacting the starting composition with the glycosyltransferase polypeptide and the non-UDP-sugar sugar donor is in a reaction medium comprising pyridoxal phosphate (PLP) and/or one or more metal ions. In some embodiments, the step of contacting the starting composition with the glycosyltransferase polypeptide and the non-UDP-sugar sugar donor is at temperature between about 0° C. and about 60° C. In other embodiments, the step of contacting the starting composition with the glycosyltransferase polypeptide and the non-UDP-sugar sugar donor is at temperature about 30° C. In other embodiments, the step of contacting the starting composition with the glycosyltransferase polypeptide and the non-UDP-sugar sugar donor is carried out in a duration of time between 1 hour and 1 week. In further embodiments, the step of contacting the starting composition with the glycosyltransferase polypeptide and the non-UDP-sugar sugar donor is carried out in a duration of time of about 24 hours.

### Two-Step Methods of Producing a Target Steviol Glycoside

In some embodiments, provided herein is a method for producing a target steviol glycoside composition, comprising the steps of: (a) providing a starting composition comprising greater than about 0.5%, about 1%, about 2%, about 3%, about 4%, about 5%, about 10%, about 20%, about 30%, about 40%, about 50%, about 60%, about 70%, about 80%, about 90%, about 92%, about 93%, about 94%, about 95%, about 96%, about 97%, about 98%, about 99%, or about 99.6% of a substrate steviol glycoside by weight on an anhydrous basis; (b) contacting the starting composition with a first glycosyltransferase polypeptide and a non-UDP-sugar sugar donor; (c) producing an intermediate composition comprising an intermediate target steviol glycoside; (d) contacting the intermediate composition with a second glycosyltransferase polypeptide and the non-UDP-sugar sugar donor; and (e) producing a target composition comprising a target steviol glycoside.

In some embodiments, provided herein is a method for producing a target steviol glycoside composition, comprising the steps of: (a) providing a starting composition comprising greater than about 0.5%, about 1%, about 2%, about 3%, about 4%, about 5%, about 10%, about 20%, about 30%, about 40%, about 50%, about 60%, about 70%, about 80%, about 90%, about 92%, about 93%, about 94%, about 95%, about 96%, about 97%, about 98%, about 99%, or about 99.6% of a substrate steviol glycoside by weight on an anhydrous basis; (b) contacting the starting composition with a first glycosyltransferase polypeptide and a first non-UDP-sugar sugar donor; (c) producing an intermediate composition comprising an intermediate target steviol glycoside; (d) contacting the intermediate composition with a second glycosyltransferase polypeptide and a second non-UDP-sugar sugar donor; and (e) producing a target composition comprising a target steviol glycoside.

In some embodiments, the first non-UDP-sugar sugar donor and the second non-UDPsugar sugar donor are identical. In other embodiments, the first non-UDP-sugar sugar donor and the second non-UDP-sugar sugar donor are different. In some embodiments, the first and/or second non-UDP-sugar sugar donor is alpha-glucose-1-phosphate, beta-glucose-1-phosphate, cellobiose, sucrose, maltose, gentiobiose, trehalose, kojibiose, nigerose, isomaltose, beta-beta-trehalose, alpha-beta-trehalose, sophorose, laminaribiose, turanose, maltulose, palatinose, gentiobiulose, nigerotriose, maltotriose, melezitose, maltotriulose, kestose, starch, cellulose, glycogen, amylose, amylopectin, dextran, dextrin, malto-dextrin, glucose syrup, cellodextrin, cyclodextrin, a non-UDP nucleotide sugar (i.e. ADP-glucose, GDP-glucose, CDP-glucose, TDP-glucose), or a steviol glycoside. In further embodiments, the first and/or second non-UDP-sugar sugar donor is not alpha-glucose-1-phosphate. In further embodiments, the first and/or second non-UDP-sugar sugar donor is not beta-glucose-1-phosphate.

In some embodiments, the first glycosyltransferase polypeptide comprises an amino acid sequence that is at least 60%, at least 65%, at least 70%, at least 75%, at least 80%, at least 85%, at least 90%, at least 95%, at least 98%, or at least 99% identical to an amino acid sequence selected from the group consisting of SEQ ID NOs: 1-128.

In some embodiments, the second glycosyltransferase polypeptide comprises an amino acid sequence that is at least 60%, at least 65%, at least 70%, at least 75%, at least 80%, at least 85%, at least 90%, at least 95%, at least 98%, or at least 99% identical to an amino acid sequence selected from the group consisting of SEQ ID NOs: 1-128.

In some embodiments, the first glycosyltransferase polypeptide and the second glycosyltransferase polypeptide are identical. In other embodiments, the first glycosyltransferase polypeptide and the second glycosyltransferase polypeptide are different.

In some embodiments, the intermediate composition comprises greater than about 5%, about 10%, about 20%, about 30%, about 40%, about 50%, about 60%, about 70%, about 80%, about 85%, about 90%, about 91%, about 92%, about 93%, about 94%, about 95%, about 96%, about 97%, about 98%, about 99%, or about 99.6% of the intermediate target steviol glycoside by weight on an anhydrous basis.

In other embodiments, the target composition comprises greater than about 0.5%, about 1%, about 2%, about 3%, about 4%, about 5%, about 10%, about 20%, about 30%, about 40%, about 50%, about 60%, about 70%, about 80%, about 85%, about 90%, about 91%, about 92%, about 93%, about 94%, about 95%, about 96%, about 97%, about 98%, about 99%, or about 99.6% of the target steviol glycoside by weight on an anhydrous basis.

In some embodiments, the substrate steviol glycoside is steviol, steviol-13-O-glucoside, steviol-19-O-glucoside, rubusoside, steviol-1,2-bioside, steviol-1,3-bioside, rubusoside, dulcoside B, dulcoside A, rebaudioside B, rebaudioside G, stevioside, rebaudioside C, rebaudioside F, rebaudioside A, rebaudioside I, rebaudioside E, rebaudioside H, rebaudioside L, rebaudioside K, rebaudioside J, rebaudioside M, rebaudioside D, rebaudioside N, rebaudioside O, rebaudioside Q, an isomer thereof, a synthetic steviol glycoside or combinations thereof. In some embodiments, the non-UDP-sugar sugar donor is alpha-glucose-1-phosphate, beta-glucose-1-phosphate, cellobiose, sucrose, maltose, gentiobiose, trehalose, kojibiose, nigerose, isomaltose, beta-beta-trehalose, alpha-beta-trehalose, sophorose, laminaribiose, turanose, maltulose, palatinose, gentiobiulose, nigerotriose, maltotriose, melezitose, maltotriulose, kestose, starch, cellulose, glycogen, amylose, amylopectin, dextran, dextrin, maltodextrin, glucose syrup, cellodextrin, cyclodextrin, a non-UDP nucleotide sugar (i.e. ADP-glucose, GDP-glucose, CDP-glucose, TDP-glucose), or a steviol glycoside. In further embodiments, the non-UDP-sugar sugar donor is not alpha-glucose-1-phosphate. In further embodiments, the non-UDP-sugar sugar donor is not beta-glucose-1-phosphate.

In some embodiments, at least about 5%, about 10%, about 20%, about 30%, about 40%, about 50%, about 60%, about 70%, about 80%, or about 90% of the substrate steviol glycoside in the starting composition is converted to the intermediate target steviol glycoside.

In some embodiments, about 30%, about 40%, about 50%, about 60%, about 70%, about 80%, or about 90% of the intermediate target steviol glycoside in the intermediate composition is converted to the target steviol glycoside.

In some embodiments, the substrate steviol glycoside is rebaudioside A, or isomers thereof; and the intermediate steviol glycoside is rebaudioside D, or isomers thereof; and the target steviol glycoside is rebaudioside M or isomers thereof. In some embodiments, the substrate steviol glycoside is rebaudioside D, or isomers thereof; and the intermediate steviol glycoside is rebaudioside M, or isomers thereof; and the target steviol glycoside is a rebaudioside Mp1 isomer.

In some embodiments, the starting composition is a Stevia rebaudiana extract. In other embodiments, the glucosyltransferase polypeptide is expressed in a host microorganism. In further embodiments, the host microorganism is E. coli, Saccharomyces sp., Aspergillus sp., Pichia sp., or Bacillus sp.

In some embodiments, the glucosyltransferase polypeptide is immobilized to a solid support. In other embodiments, the solid support is derivatized cellulose or glass, ceramics, methacrylate, styrene, acrylic, metal oxides, or membranes.

In some embodiments, the step of contacting the starting composition with the first glycosyltransferase polypeptide and the non-UDP-sugar sugar donor is in a reaction medium comprising pyridoxal phosphate (PLP) and/or one or more metal ions. In some embodiments, the step of contacting the starting composition with the first glycosyltransferase polypeptide and the non-UDP-sugar sugar donor is at temperature between about 0° C. and about 60° C. In other embodiments, the step of contacting the starting composition with the first glycosyltransferase polypeptide and the non-UDP-sugar sugar donor is at temperature about 30° C. In other embodiments, the step of contacting the starting composition with the first glycosyltransferase polypeptide and the non-UDP-sugar sugar donor is carried out in a duration of time between 1 hour and 1 week. In further embodiments, the step of contacting the starting composition with the first glycosyltransferase polypeptide and the non-UDP-sugar sugar donor is carried out in a duration of time of about 24 hours.

In some embodiments, the step of contacting the intermediate composition with the second glycosyltransferase polypeptide and the non-UDP-sugar sugar donor is in a reaction medium comprising pyridoxal phosphate (PLP) and/or one or more metal ions. In some embodiments, the step of contacting the intermediate composition with the second glycosyltransferase polypeptide and the non-UDP-sugar sugar donor is at temperature between about 0° C. and about 60° C. In other embodiments, the step of contacting the intermediate composition with the second glycosyltransferase polypeptide and the non-UDP-sugar sugar donor is at temperature about 30° C. In further embodiments, the step of contacting the intermediate composition with the second glycosyltransferase polypeptide and the non-UDP-sugar sugar donor is carried out in a duration of time between 1 hour and 1 week. In further embodiments, the step of contacting the intermediate composition with the second glycosyltransferase polypeptide and the non-UDP-sugar sugar donor is carried out in a duration of time of about 24 hours.

Recombinant Glycosyltransferase

In some embodiments, provided herein is a recombinant glycosyltransferase polypeptide comprising an amino acid sequence that is at least 60%, at least 65%, at least 70%, at least 75%, at least 80%, at least 85%, at least 90%, at least 95%, at least 98%, or at least 99% identical to an amino acid sequence selected from the group consisting of SEQ ID NOs: 1-128. In another embodiment, the glucosyltransferase polypeptide is a polypeptide sequence that is at least 60%, 65%, 70%, 75%, 80%, 85%, 86%, 87%, 88%, 89%, 90%, 91%, 92%, 93%, 94%, 95%, 96%, 97%, 98%, 99%, or more sequence identity to one of SEQ ID NOs: 1, 19, 29, 32, 41, 42, 43, 44, 45, 46, 47, 48, 49, 50, 51, 52, 53, 54, 55, 56, 58, 61, 65, 67, 68, 81, 87, 90, 91, 92, 93, 94, 95, 96, 97, 98, 99, 100, 101, 102, 105, 106, 116, 120, 122, and 127. In other embodiments, the recombinant glycosyltransferase polypeptide comprises an amino acid sequence that is at least 60%%, 65%, 70%, 75%, 80%, 85%, 86%, 87%, 88%, 89%, 90%, 91%, 92%, 93%, 94%, 95%, 96%, 97%, 98%, 99% identical to identical to an amino acid sequence selected from the group consisting of SEQ ID NOs: 2, 3, 22, 29, 39, 41, 42, 45, 49, 50, 51, 52, 53, 54, 55, 56, 62, 63, 72, 87, 90, 91, 94, 97, 98, 99, 100, 101, 102, 109, 110, 111, 112, 113, 115, 117, 118, 119, 121, 123, 124, 125, 126, and 128.

In some embodiments, the recombinant glycosyltransferase polypeptide comprises an amino acid sequence selected from the group consisting of SEQ ID NOs: 1-128. In other embodiments, the recombinant glycosyltransferase polypeptide comprises an amino acid sequence selected from the group consisting of SEQ ID NOs: 2, 3, 22, 29, 39, 41, 42, 45, 49, 50, 51, 52, 53, 54, 55, 56, 62, 63, 72, 87, 90, 91, 94, 97, 98, 99, 100, 101, 102, 109, 110, 111, 112, 113, 115, 117, 118, 119, 121, 123, 124, 125, 126, and 128.

In some embodiments, the glycosyltransferase polypeptide further comprises a tag amino acid sequence. In some embodiments, the tag amino acid sequence is His6.

In some embodiments, the recombinant glycosyltransferase polypeptide is capable of transferring a sugar moiety to a substrate steviol glycoside. In some embodiments, the substrate steviol glycoside is steviol, steviol-13-O-glucoside, steviol-19-O-glucoside, rubusoside, steviol-1,2-bioside, steviol-1,3-bioside, rubusoside, dulcoside B, dulcoside A, rebaudioside B, rebaudioside G, stevioside, rebaudioside C, rebaudioside F, rebaudioside A, rebaudioside I, rebaudioside E, rebaudioside H, rebaudioside L, rebaudioside K, rebaudioside J, rebaudioside M, rebaudioside D, rebaudioside N, rebaudioside O, rebaudioside Q, an isomer thereof, a synthetic steviol glycoside or combinations thereof. In other embodiments, the substrate steviol glycoside is rebaudioside A, or an isomer thereof. In further embodiments, the substrate steviol glycoside is rebaudioside D, or an isomer thereof

In some embodiments, the non-UDP-sugar sugar donor is alpha-glucose-1-phosphate, beta-glucose-1-phosphate, cellobiose, sucrose, maltose, gentiobiose, trehalose, kojibiose, nigerose, isomaltose, beta-beta-trehalose, alpha-beta-trehalose, sophorose, laminaribiose, turanose, maltulose, palatinose, gentiobiulose, nigerotriose, maltotriose, melezitose, maltotriulose, kestose, starch, cellulose, glycogen, amylose, amylopectin, dextran, dextrin, maltodextrin, glucose syrup, cellodextrin, cyclodextrin, a non-UDP nucleotide sugar (i.e. ADP-glucose, GDP-glucose, CDP-glucose, TDP-glucose), or a steviol glycoside.

In some embodiments, the recombinant glycosyltransferase polypeptide is expressed in a host microorganism. In other embodiments, the host microorganism is E. coli, Saccharomyces sp., Aspergillus sp., Pichia sp., or Bacillus sp.

In some embodiments, the recombinant glycosyltransferase polypeptide is immobilized to a solid support. In other embodiments, the solid support is derivatized cellulose, glass, ceramic, methacrylate, styrene, acrylic, a metal oxide, or a membrane.

### Modified Microorganism

In some embodiments, provided herein is a modified microorganism expressing a glycosyltransferase polypeptide comprises an amino acid sequence that is at least 60%, at least 65%, at least 70%, at least 75%, at least 80%, at least 85%, at least 90%, at least 95%, at least 98%, or at least 99% identical to an amino acid sequence selected from the group consisting of SEQ ID NOs: 1-128, or combinations thereof, particularly SEQ ID NOs 121 and 128. In another embodiment, the glucosyltransferase polypeptide is a polypeptide sequence that is at least 60%, 65%, 70%, 75%, 80%, 85%, 86%, 87%, 88%, 89%, 90%, 91%, 92%, 93%, 94%, 95%, 96%, 97%, 98%, 99%, or more sequence identity to one of SEQ ID NOs: 1, 19, 29, 32, 41, 42, 43, 44, 45, 46, 47, 48, 49, 50, 51, 52, 53, 54, 55, 56, 58, 61, 65, 67, 68, 81, 87, 90, 91, 92, 93, 94, 95, 96, 97, 98, 99, 100, 101, 102, 105, 106, 116, 120, 122, and 127. In other embodiments, the glycosyltransferase polypeptide comprises an amino acid sequence that is at least 60%, 65%, 70%, 75%, 80%, 85%, 86%, 87%, 88%, 89%, 90%, 91%, 92%, 93%, 94%, 95%, 96%, 97%, 98%, 99%, or more sequence identity to an amino acid sequence selected from the group consisting of SEQ ID NOs: 2, 3, 22, 29, 39, 41, 42, 45, 49, 50, 51, 52, 53, 54, 55, 56, 62, 63, 72, 87, 90, 91, 94, 97, 98, 99, 100, 101, 102, 109, 110, 111, 112, 113, 115, 117, 118, 119, 121, 123, 124, 125, 126, and 128.

In some embodiments, provided herein is a method for transferring a sugar moiety to a substrate steviol glycoside, the method comprising contacting the substrate steviol glycoside with the modified microorganism and a non-UDP-sugar sugar donor.

In some embodiments, provided herein is a method for producing a target steviol glycoside composition, comprising the steps of: (1) providing a starting composition comprising greater than about 0.5%, about 1%, about 2%, about 3%, about 4%, about 5%, about 10%, about 20%, about 30%, about 40%, about 50%, about 60%, about 70%, about 80%, about 90%, about 92%, about 93%, about 94%, about 95%, about 96%, about 97%, about 98%, about 99%, or about 99.6% of a substrate steviol glycoside by weight on an anhydrous basis; (2) contacting the starting composition with the modified microorganism and a non-UDP-sugar sugar donor; and (3) producing a target composition comprising a target steviol glycoside.

In some embodiments, the glycosyltransferase polypeptide comprises an amino acid sequence selected from the group consisting of SEQ ID NOs: 1-128. In other embodiments, the glycosyltransferase polypeptide comprises an amino acid sequence selected from the group consisting of SEQ ID NOs: 2, 3, 22, 29, 39, 41, 42, 45, 49, 50, 51, 52, 53, 54, 55, 56, 62, 63, 72, 87, 90, 91, 94, 97, 98, 99, 100, 101, 102, 109, 110, 111, 112, 113, 115, 117, 118, 119, 121, 123, 124, 125, 126, and 128. In some embodiments, the glycosyltransferase polypeptide is capable of transferring a sugar moiety to a substrate steviol glycoside.

In some embodiments, the glycosyltransferase polypeptide further comprises a tag amino acid sequence. In some embodiments, the tag amino acid sequence is His6.

In some embodiments, the substrate steviol glycoside is steviol, steviol-13-O-glucoside, steviol-19-O-glucoside, rubusoside, steviol-1,2-bioside, steviol-1,3-bioside, rubusoside, dulcoside B, dulcoside A, rebaudioside B, rebaudioside G, stevioside, rebaudioside C, rebaudioside F, rebaudioside A, rebaudioside I, rebaudioside E, rebaudioside H, rebaudioside L, rebaudioside K, rebaudioside J, rebaudioside M, rebaudioside D, rebaudioside N, rebaudioside O, rebaudioside Q, an isomer thereof, a synthetic steviol glycoside or combinations thereof. In other embodiments, the substrate steviol glycoside is rebaudioside A, or an isomer thereof. In further embodiments, the substrate steviol glycoside is rebaudioside D, or an isomer thereof. In some embodiments, the non-UDP-sugar sugar donor is alpha-glucose-1-phosphate, beta-glucose-1-phosphate, cellobiose, sucrose, maltose, gentiobiose, trehalose, kojibiose, nigerose, isomaltose, beta-beta-trehalose, alpha-beta-trehalose, sophorose, laminaribiose, turanose, maltulose, palatinose, gentiobiulose, nigerotriose, maltotriose, melezitose, maltotriulose, kestose, starch, cellulose, glycogen, amylose, amylopectin, dextran, dextrin, maltodextrin, glucose syrup, cellodextrin, or cyclodextrin, a non-UDP nucleotide sugar (i.e. ADP-glucose, GDP-glucose, CDP-glucose, TDP-glucose), or a steviol glycoside.

In some embodiments, the modified microorganism is E. coli, Saccharomyces sp., Aspergillus sp., Pichia sp., or Bacillus sp. In other embodiments, the glucosyltransferase polypeptide is immobilized to a solid support. In further embodiments, the solid support is derivatized cellulose, glass, ceramic, methacrylate, styrene, acrylic, a metal oxide, or a membrane.

### EXAMPLES

### Example 1: In-Vivo Production of Glucosyltransferases

Polynucleotides encoding amino acid sequences SEQ ID NO: 1-71 and SEQ ID NO: 109-122 were synthesized (Twist Bioscience) and inserted into the pARZ4 expression vector, generating the following recombinant vectors: pA10225, pA10143, pA10080, pA10135, pA10139, pA10170, pA10122, pA10260, pA10113, pA10180, pA10163, pA10141, pA10181, pA10231, pA10243, pA10172, pA10108, pA10263, pA10251, pA10118, pA10223, pA10082, pA10157, pA10206, pA10166, pA10192, pA10081, pA10204, pA10085, pA10177, pA10121, pA10105, pA10209, pA10216, pA10123, pA10221, pA10140, pA10134, pA10109, pA10131, pA10174, pA10154, pA10152, pA10151, pA10076, pA10155, pA10072, pA10265, pA10074, pA10075, pA10194, pA10176, pA10078, pA10073, pA10147, pA10158, pA10116, pA10262, pA10203, pA10266, pA10149, pA10197, pA10195, pA10212, pA10115, pA10211, pA12546, pA10114, pA10162, pA10126, pA10125, pA10112, pA10213, pA10175, pA10117, pA10259, pA10190, pA10098, pA10188, pA10189, pA10160, pA10084, pA10185, pA10273, pA10224. The recombinant vectors were used in a heat shock method to transform E. coli NEBT7EL (New England Biolabs), thereby preparing recombinant microorganisms.

The transformed recombinant microorganism was inoculated to 1 ml LB-kanamycin medium, cultured by shaking at 37° C. overnight. The culture was inoculated to 5 ml TB-kanamycin medium and grown for 2 hours at 37° C., followed by 25° C. for 1 hour. The culture was induced with 50 uL 50 mM IPTG and grown overnight. Finally, the culture was centrifuged at top-speed for 5-minutes and stored at −80° C.

### Example 2: Purification of Glucosyltransferases

The microorganisms created in Example 1 were dissolved in a lysis buffer (lysozyme, DNAseI, Bugbuster, 300 mL 20 mM PO₄ pH 7.5, 500 mM NaCl, and 20 mM Imidazole). Two to three glass beads were added to each well and were disrupted by shaking at 25° C. and 220 rpm for 30 minutes. The disrupted liquid was centrifuged at 2200× g for 6-10 minutes. The obtained supernatant was loaded onto a Ni-NTA plate and shaken for 10 minutes at room temperature. The plate was centrifuged for 4 minutes at 100× g followed by two washes of 500 uL binding buffer (300 mL 20 mM PO₄ pH 7.5, 500 mM NaCl, 20 mM Imidazole) and two minute centrifugation (500× g). The proteins were eluted with 150 uL elution buffer (15 mL 20 mM PO₄ pH 7.5.5, 500 mM NaCl, 500 mM Imidazole) and shaken for 1 minute at 0.25 maximum shaking speed followed by centrifugation for 2 minutes at 500× g. The recovered protein was desalted into a buffer solution for enzyme activity evaluation (20 mM HEPES, 50 mM NaCl, pH 7.5)

### Example 3: Measure Glucosyltransferase Activity with RebA Substrate and Alpha-Glucose-1-Phosphate Sugar Donor

Purified enzyme from Example 2 was incubated in 100 mM of appropriate buffer (acetate buffer, pH 5; citrate buffer, pH 6.5; HEPES buffer, pH 7.5; or HEPES buffer, pH 8) with 1 mM RebA, 5 mM alpha-glucose-1-phosphate (AG1P), 50 mM NaCl and 1 mM of appropriate cofactor (PLP, CaCl₂, MgCl₂ or MnCl₂) overnight at 30° C. Product rebaudiosides were separated by reverse phase chromatography. Detection was accomplished by an Agilent 6545 QTOF mass spectrophotometer in negative mode. Rebaudioside D and rebaudioside M were identified by comparison of retention times to known standards. Isomers were identified by relative retention time to the standards. Glucosyltransferases were identified that could make rebD isomers and rebM isomers (Table 3).

### Example 4: Measure Glucosyltransferase Activity with RebA Substrate and Cellobiose Sugar Donor

Purified enzyme from Example 2 was incubated in 100 mM of appropriate buffer (acetate buffer, pH 5; citrate buffer, pH 6.5; HEPES buffer, pH 7.5; or HEPES buffer, pH 8) with 1 mM rebA, 5 mM cellobiose, 50 mM NaCl and 1 mM of appropriate cofactor (PLP, CaCl₂, MgCl₂ or MnCl₂) overnight at 30° C.

Enzymatic reactions were analyzed for rebaudioside content by UPLC chromatography coupled with Q-TOF MS detection. The products of enzymatic reactions were separated by UPLC on a 150 mm HSS T3 chromatography column using mobile phases of 0.1% formic acid in H₂O and 0.1% formic acid in acetonitrile (Table 1). Chromatographically separated products of enzymatic reactions were detected using an Agilent 6545 quadrupole time-of-flight mass spectrometer in negative mode (Table 2). Rebaudioside identification of RebD and RebM were accomplished by comparison to commercially purchased analytical standards (Chromadex). Commercial standards were dissolved in 90% water, 10% methanol. Rebaudioside isomers were identified by relative retention time to the standards. Glucosyltransferases were identified that could make rebD isomers and rebM isomers (Table 3).

TABLE 1
Chromatography gradient. 4.5 minute separation
of rebaudiosides. Flowrate: 0.5 ml/min

| | % A | % B |
|---|---|---|
| Time | 0.1% formic acid in H2O | 0.1% formic acid in acetonitrile |
| 0 | 80 | 20 |
| 2 | 60 | 40 |
| 2.4 | 0 | 100 |
| 2.9 | 80 | 200 |
| 4.5 | stop | Stop |

TABLE 2
Mass Spectrophotometer Settings
ESI Source Settings

| | | |
|---|---|---|
| Gas Temp | 300 | C. |
| Drying Gas | 13 | l/min |
| Nebulizer | 25 | psig |
| Sheath Gas Temp | 400 | C. |
| Sheath Gas Flow | 12 | l/min |
| Vcap | 3000 | V |
| Nozzle Voltage | 1500 | V |

MS TOF Settings

| | | |
|---|---|---|
| Fragmentor | 80 | V |
| Skimmer | 50 | V |
| OCT 1 RF Vpp | 750 | V |

m/z

| | |
|---|---|
| RebD Isomers in Negative Mode | 1127.4763 |
| RebM Isomers in Negative Mode | 1289.5400 |
| RebMp1 Isomers in Negative Mode | 1451.5820 |

### Example 5: Measure Glucosyltransferase Activity with RebA Substrate and Sucrose Sugar Donor

Purified enzyme from Example 2 was incubated in 100 mM of appropriate buffer (acetate buffer, pH 5; citrate buffer, pH 6.5; HEPES buffer, pH 7.5; or HEPES buffer, pH 8) with 1 mM rebA, 5 mM sucrose, 50 mM NaCl and 1 mM of appropriate cofactor (PLP, CaCl₂, MgCl₂ or MnCl₂) overnight at 30° C. Product rebaudiosides were measured as in Example 4. Glucosyltransferases were identified that could make rebD isomers and rebM isomers (Table 3).

### Example 6: Measure Glucosyltransferase Activity with RebA Substrate and Maltose Sugar Donor

Purified enzyme from Example 2 was incubated in 100 mM of appropriate buffer (acetate buffer, pH 5; citrate buffer, pH 6.5; HEPES buffer, pH 7.5; or HEPES buffer, pH 8) with 1 mM rebA, 5 mM maltose, 50 mM NaCl and 1 mM of appropriate cofactor (PLP, CaCl₂, MgCl₂ or MnCl₂) overnight at 30° C. Product rebaudiosides were measured as in Example 4. Glucosyltransferases were identified that could make rebD isomers and rebM isomers (Table 3).

### Example 7: Measure Glucosyltransferase Activity with RebA Substrate and Gentiobiose Sugar Donor

Purified enzyme from Example 2 was incubated in 100 mM of appropriate buffer (acetate buffer, pH 5; citrate buffer, pH 6.5; HEPES buffer, pH 7.5; or HEPES buffer, pH 8) with 1 mM rebA, 5 mM gentiobiose, 50 mM NaCl and 1 mM of appropriate cofactor (PLP, CaCl₂, MgCl₂ or MnCl₂) overnight at 30° C. Product rebaudiosides were measured as in Example 4.Glucosyltransferases were identified that could make rebD isomers and rebM isomers (Table 3).

TABLE 3
Summary of steviol glycoside products made by each glucosyltransferase
when reacted with rebA and the specified sugar donor.

| | | | Products | | | | | | | | | | |
|---|---|---|---|---|---|---|---|---|---|---|---|---|---|
| | | | RebD Isomers | | | | | RebM Isomers | | | | | |
| Plasmid ID | Seq No | SugarDonor | 0.77 | 1.07 | 1.21 | 1.25 | 1.29 | 0.66 | 0.92 | 1.06 | 1.11 | 1.17 | 1.51 |
| pA10072 | 47 | 5ucrose | | + | | | | | | | | | |
| pA10073 | 54 | Maltose | | | + | | + | | | + | + | + | |
| pA10074 | 49 | Maltose | | + | + | | + | | | | + | + | |
| pA10075 | 50 | Maltose | | + | + | | + | | | + | + | + | |
| pA10076 | 45 | Maltose | | + | + | | + | | + | | + | + | |
| pA10078 | 53 | Gentiobiose | | | + | | + | | | + | + | + | |
| pA10078 | 53 | Maltose | | | + | | + | | | + | + | + | |
| pA10085 | 29 | Cellobiose | | | | | | + | | | | | |
| pA10085 | 29 | Gentiobiose | | | | | | + | | | | | |
| pA10085 | 29 | Maltose | | | | | | + | | | | | |
| pA10085 | 29 | Sucrose | | | | | | + | | | | | |
| pA10105 | 32 | AG1P | | | | | | | | | | | + |
| pA10114 | 68 | Gentiobiose | | | | | + | | | | | + | |
| pA10114 | 68 | Maltose | | | | | + | | | | | | |
| pA10115 | 65 | Gentiobiose | | | | + | | | | | | | |
| pA10147 | 55 | AG1P | | | | | + | | | | | | |
| pA10147 | 55 | BG1P | | | | | + | | | | | | |
| pA10147 | 55 | Cellobiose | | | | | + | | | | | | |
| pA10147 | 55 | Gentiobiose | | | | | + | | | | | | |
| pA10147 | 55 | Maltose | | | | | + | | | | | | |
| pA10147 | 55 | Sucrose | | | | | + | | | | | | |
| pA10149 | 61 | Sucrose | | + | | | | | | | | | |
| pA10151 | 44 | AG1P | | + | | | | | | | | | |
| pA10151 | 44 | Maltose | | + | | + | | | | | | | |
| pA10151 | 44 | Sucrose | | + | | + | | | | | | | |
| pA10152 | 43 | AG1P | | + | | | | | | | | | |
| pA10152 | 43 | Maltose | | + | | | | | | | | | |
| pA10152 | 43 | Sucrose | | + | | + | | | + | | | | |
| pA10154 | 42 | AG1P | | + | | | | | | | | | |
| pA10154 | 42 | Maltose | | + | | | | | | | | | |
| pA10154 | 42 | Sucrose | | + | | + | | | + | | | | |
| pA10155 | 46 | Maltose | | + | | + | | | | | | | |
| pA10158 | 56 | Gentiobiose | | | | | | | | | | | + |
| pA10174 | 41 | Maltose | | | + | | + | | | + | + | + | |
| pA10176 | 52 | Maltose | | + | + | + | + | | | | | + | |
| pA10185 | 120 | AG1P | + | | | | | | | | | | |
| pA10188 | 116 | BG1P | | | | | + | | | | | | |
| pA10194 | 51 | Gentiobiose | | | + | | + | | | + | + | + | |
| pA10194 | 51 | Maltose | | + | + | | + | | | + | + | + | |
| pA10224 | 122 | AG1P | | | | | | | | | | + | |
| pA10225 | 1 | Maltose | + | | | | | | | | | | |
| pA10251 | 19 | AG1P | | | | | | | | | | | + |
| pA10262 | 58 | Sucrose | | | | | + | | | | | | |
| pA10265 | 48 | Sucrose | | + | | | | | | | | | |
| pA12546 | 67 | Maltose | | | | | + | | | | | + | |

### Example 8: Measure Glucosyltransferase Activity with RebA Substrate and Beta-glucose1-Phosphate Sugar Donor

Purified enzyme from Example 2 was incubated in 100 mM of appropriate buffer (acetate buffer, pH 5; citrate buffer, pH 6.5; HEPES buffer, pH 7.5; or HEPES buffer, pH 8) with 1 mM rebA, 5 mM beta-glucose-1-phosphate (BG1P), 50 mM NaCl and 1 mM of appropriate cofactor (PLP, CaCl₂, MgCl₂ or MnCl₂) overnight at 30° C. Product rebaudiosides were measured as in Example 4. Glucosyltransferases were identified that could make rebD isomers and rebM isomers (Table 3).

### Example 9: Measure Glucosyltransferase Activity with RebD Substrate and Alpha-Glucose-1-Phosphate Sugar Donor

Purified enzyme from Example 2 was incubated in 100 mM of appropriate buffer (acetate buffer, pH 5; citrate buffer, pH 6.5; HEPES buffer, pH 7.5; or HEPES buffer, pH 8) with 1 mM rebD, 5 mM alpha-glucose-1-phosphate, 50 mM NaCl 1 mM of appropriate cofactor (PLP, CaCl₂, MgCl₂ or MnCl₂) and 2% DMSO overnight at 30° C. Product rebaudiosides were measured as in Example 4.Glucosyltransferases were identified that could make rebM isomers and rebMp1 isomers (Table 4).

### Example 10: Measure Glucosyltransferase Activity with RebD Substrate and Cellobiose Sugar Donor

Purified enzyme from Example 2 was incubated in 100 mM of appropriate buffer (acetate buffer, pH 5; citrate buffer, pH 6.5; HEPES buffer, pH 7.5; or HEPES buffer, pH 8) with 1 mM rebD, 5 mM cellobiose, 50 mM NaCl, 1 mM of appropriate cofactor (PLP, CaCl₂, MgCl₂ or MnCl₂) 2% DMSO overnight at 30° C. Product rebaudiosides were measured as in Example 4.Glucosyltransferases were identified that could make rebM isomers and rebMp1 isomers (Table 4).

### Example 11: Measure Glucosyltransferase Activity with RebD Substrate and Sucrose Sugar Donor

Purified enzyme from Example 2 was incubated in 100 mM of appropriate buffer (acetate buffer, pH 5; citrate buffer, pH 6.5; HEPES buffer, pH 7.5; or HEPES buffer, pH 8) with 1 mM rebD, 5 mM sucrose, 50 mM NaCl, 1 mM of appropriate cofactor (PLP, CaCl₂, MgCl₂ or MnCl₂) and 2% DMSO overnight at 30° C. Product rebaudiosides were measured as in Example 4.Glucosyltransferases were identified that could make rebM isomers and rebMp1 isomers (Table 4).

### Example 12: Measure Glucosyltransferase Activity with RebD Substrate and Maltose Sugar Donor

Purified enzyme from Example 2 was incubated in 100 mM of appropriate buffer (acetate buffer, pH 5; citrate buffer, pH 6.5; HEPES buffer, pH 7.5; or HEPES buffer, pH 8) with 1 mM rebD, 5 mM maltose, 50 mM NaCl, 1 mM of appropriate cofactor (PLP, CaCl₂, MgCl₂ or MnCl₂) and 2% DMSO overnight at 30° C. Product rebaudiosides were measured as in Example 4. Glucosyltransferases were identified that could make rebM isomers and rebMp1 isomers (Table 4).

TABLE 4
Summary of steviol glycoside products made by each glucosyltransferase
when reacted with Reb D and the specified sugar donor.

| | | | Products | | | | | | | | | |
|---|---|---|---|---|---|---|---|---|---|---|---|---|
| | | | RebM Isomers | | | | RebMp1 Isomers | | | | | |
| Plasmid ID | SeqNo | SugarDonor | 0.66 | 0.8 | 0.92 | rebM | 0.76 | 0.82 | 0.88 | 0.94 | 1.09 | 1.66 |
| pA10073 | 54 | Maltose | | + | + | | + | + | + | + | | |
| pA10074 | 49 | Maltose | | + | + | | | | | | | |
| pA10075 | 50 | Maltose | | + | + | | + | + | | + | | |
| pA10076 | 45 | Maltose | | + | + | | | | | | | |
| pA10078 | 53 | Maltose | | + | + | | + | + | | + | | |
| pA10080 | 3 | AG1P | | + | | | | + | | | | |
| pA10082 | 22 | AG1P | | + | | | | | | | | |
| pA10084 | 119 | Maltose | | + | + | | | | | | | |
| pA10084 | 119 | Sucrose | | + | + | | | | | | | |
| pA10085 | 29 | Cellobiose | + | | | | | | | | | |
| pA10098 | 115 | AG1P | | | | | | | | | + | |
| pA10098 | 115 | Cellobiose | | | | | | | | | + | |
| pA10109 | 39 | AG1P | | + | | | | | | | | |
| pA10112 | 109 | Maltose | | + | | | | | | | | |
| pA10117 | 112 | Maltose | | + | | | | | | | | |
| pA10143 | 2 | AG1P | | + | | | | | | | | |
| pA10143 | 2 | Cellobiose | | + | | | | | | | | |
| pA10143 | 2 | Maltose | | + | | | | | | | | |
| pA10143 | 2 | Sucrose | | + | | | | | | | | |
| pA10147 | 55 | AG1P | | | | + | | | | | | |
| pA10147 | 55 | Cellobiose | | | | + | | | | | | |
| pA10147 | 55 | Maltose | | | | + | | | | | | |
| pA10147 | 55 | Sucrose | | | | + | | | | | | |
| pA10154 | 42 | AG1P | | + | | | | | | | | |
| pA10158 | 56 | AG1P | | + | | | + | + | | | | |
| pA10160 | 118 | Maltose | | + | | | | | | | | |
| pA10174 | 41 | Maltose | | + | + | | + | + | | + | | |
| pA10175 | 111 | Maltose | | + | | | | | | | | |
| pA10176 | 52 | Maltose | | + | | | | | | | | |
| pA10189 | 117 | Maltose | | + | | | | | | | | |
| pA10194 | 51 | Maltose | | + | + | | + | + | | + | | |
| pA10195 | 63 | Maltose | | + | | | + | | | | | |
| pA10197 | 62 | Maltose | | + | | | + | | | | | |
| pA10213 | 110 | AG1P | | | | | | | | | | |
| pA10259 | 113 | Maltose | | + | + | | + | + | | + | | |
| pA10273 | 121 | Cellobiose | | | | | | | | | | + |
| pA10273 | 121 | Maltose | | + | | | | | | | | |

### Example 13: Validation of Top Glucosyltransferases with RebA Substrate

The most active enzymes from the glycosyltransferase library screening (Examples 3-12) were chosen for further validation. The chosen enzymes were purified as in Example 2. The enzymes were assayed in 100 mM of appropriate buffer (acetate buffer, pH 5; citrate buffer, pH 6.5; HEPES buffer, pH 7.5; or HEPES buffer, pH 8) with 1 mM rebA, 5 mM sugar donor (maltose, sucrose, cellobiose, alpha-glucose-1-phosphate, or gentiobiose), 50 mM NaCl, 1 mM of appropriate cofactor (PLP, CaCl₂, MgCl₂ or MnCl₂) and 2% DMSO overnight at 30° C. Product rebaudiosides were measured as in Example 4. Validated glucosyltransferases that make rebD isomers, rebM isomers, and rebMp1 isomers are shown in Table 5.

TABLE 5
Summary of steviol glycoside products made by each glucosyltransferase
when reacted with Reb A and the specified sugar donor.

| | | | Products | | | | | | | | | | | | |
|---|---|---|---|---|---|---|---|---|---|---|---|---|---|---|---|
| | | | RebD Isomers | | | | | RebM Isomers | | | | | RebMp1 Isomers | | |
| Plasmid ID | Seq No | SugarDonor | 0.77 | 1.07 | 1.21 | 1.25 | 1.29 | 0.66 | 0.92 | 1.06 | 1.11 | 1.17 | 1.09 | 1.14 | 1.19 |
| pA10072 | 47 | Sucrose | | + | | | | | | | | | | | |
| pA10073 | 54 | Maltose | | | + | | + | | | + | + | + | | + | + |
| pA10074 | 49 | Maltose | | + | + | | + | | | | + | + | | | |
| pA10075 | 50 | Maltose | | + | + | | + | | | + | + | + | + | + | + |
| pA10076 | 45 | Maltose | | + | + | | + | | | + | + | | | | |
| pA10078 | 53 | Maltose | | | + | | + | | | + | + | + | | + | + |
| pA10085 | 29 | Cellobiose | | | | | | + | | | | | | | |
| pA10085 | 29 | Sucrose | + | | | | + | + | | | | | | | |
| pA10147 | 55 | AG1P | | | | | + | | | | | | | | |
| pA10147 | 55 | Cellobiose | | | | | + | | | | | | | | |
| pA10147 | 55 | Gentiobiose | | | | | + | | | | | | | | |
| pA10147 | 55 | Maltose | | | | | + | | | | | | | | |
| pA10147 | 55 | Sucrose | | | | | + | | | | | | | | |
| pA10151 | 44 | AG1P | | + | | | + | | | | | | | | |
| pA10151 | 44 | Maltose | | + | | + | | | | | | | | | |
| pA10151 | 44 | Sucrose | | + | + | + | | | | | | | | | |
| pA10152 | 43 | AG1P | | + | | | + | | | | | | | | |
| pA10152 | 43 | Maltose | | + | | | + | | | | | | | | |
| pA10152 | 43 | Sucrose | | + | + | | | | + | | | | | | |
| pA10154 | 42 | AG1P | | + | | | | | + | | | | | | |
| pA10154 | 42 | Maltose | | | | | + | | | | | | | | |
| pA10154 | 42 | Sucrose | | + | + | | | | + | | | | | | |
| pA10155 | 46 | Maltose | | + | + | + | | | | | | | | | |
| pA10174 | 41 | Maltose | | | + | | + | | | + | | + | + | | + |
| pA10176 | 52 | Maltose | | + | + | + | + | | | | + | + | | | |
| pA10194 | 51 | Maltose | | + | + | | + | | | + | + | + | + | + | + |
| pA10225 | 1 | Cellobiose | + | | | | | | | | | | | | |
| pA10262 | 58 | AG1P | | | | | + | | | | | | | | |
| pA10262 | 58 | Sucrose | | | | | + | | | | | | | | |

### Example 14: Conversion of RebA to RebD_1.29 with RebA Sugar Donor

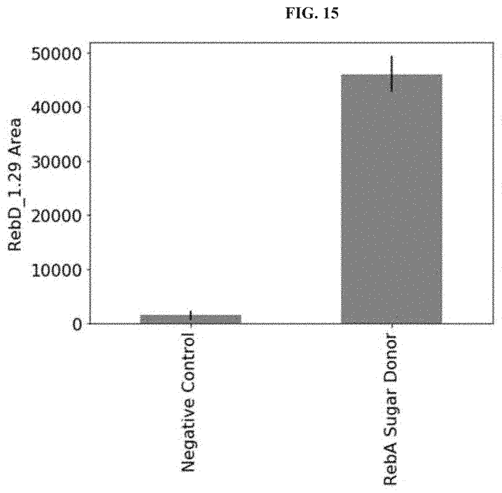

The polypeptide encoded by pA10147 (SEQ NO: 55) was expressed and purified as in Examples 1 and 2. The enzyme was assayed for activity to use RebA as a sugar donor to transfer a glucose molecule from RebA onto another RebA molecule. The enzyme was assayed in 100 mM HEPES buffer, pH 7.5 with 1 mM RebA, 50 mM NaCl, and 1 mM of MgCl₂ and 0.8% DMSO overnight at 30° C. Product rebaudiosides were measured as in Example 4. The pA10147 enzyme produced the RebD_1.29 at quantities significantly greater than background (FIG. 15).

### Example 15: Conversion of RebD to RebM with RebD Sugar Donor

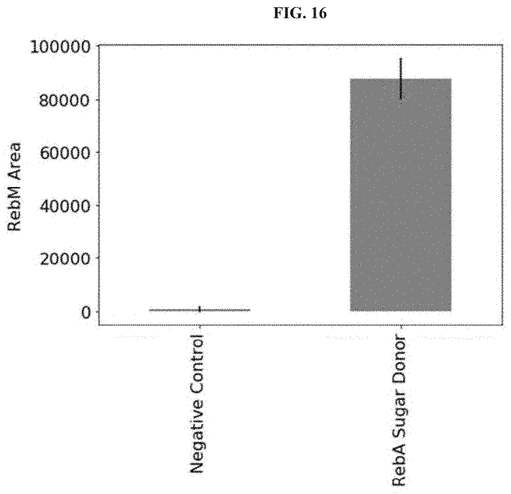

The polypeptide encoded by pA10147 (SEQ NO: 55) was expressed and purified as in Examples 1 and 2. The enzyme was assayed for activity to use RebD as a sugar donor to transfer a glucose molecule from RebD onto another RebD molecule. The enzyme was assayed in 100 mM HEPES buffer, pH 7.5 with 0.75 mM RebD, 50 mM NaCl, and 1 mM of MgCl₂ and 2% DMSO overnight at 30° C. Product rebaudiosides were measured as in Example 4. The pA10147 enzyme produced RebM at quantities significantly greater than background (FIG. 16).

### Example 16: Rebaudioside Production from RebA Feed with Maltose, Sucrose and Alpha-Glucose-1-phosphate Sugar Donors

The polypeptides encoded by the plasmids pA10151, pA10152, pA10154, pA10072, pA10176, pA10074, pA10075, pA10076, pA10155, pA10174, pA10073, pA10078, pA10194 were expressed and purified as in Example 1 and 2. The enzymes were assayed in 100 mM of appropriate buffer (sodium acetate buffer, pH 5 ; HEPES buffer, pH 7.5) with 1 mM rebA, 5 mM sugar donor (maltose, sucrose, or alpha-glucose-1-phosphate), 50 mM NaCl, 1 mM of CaCl₂ and 0.8% DMSO overnight at 30° C.

Enzymatic reactions were analyzed for rebaudioside content by UPLC chromatography coupled with Q-TOF MS detection. The products of enzymatic reactions were separated by UPLC on a 150 mm HSS T3 chromatography column using mobile phases of 0.1% formic acid in H₂O and 0.1% formic acid in acetonitrile (Table 6). Chromatographically separated products of enzymatic reactions were detected using an Agilent 6545 quadrupole time-of-flight mass spectrometer in negative mode (Table 2). Rebaudioside identification of RebD and RebM was accomplished by comparison to commercially purchased analytical standards (Chromadex). Commercial standards were dissolved in 90% water, 10% methanol. Rebaudioside isomers were identified by relative retention time to the standards. Glucosyltransferase activity for rebD isomers, rebM isomers, and rebMp1 isomers are shown in Table 7.

TABLE 6
Chromatography gradient. 20 minute separation
of rebaudiosides. Flowrate: 0.4 ml/min

| | % A: | % B: |
|---|---|---|
| Time (min) | 0.1% formic acid in H2O | 0.1% formic acid in acetonitrile |
| 0 | 80% | 20% |
| 17 | 66.6% | 33.4% |
| 17.25 | 20% | 80% |
| 18 | 20% | 80% |
| 18.5 | 80% | 20% |
| 20 | Stop | stop |

TABLE 7
Summary of steviol glycoside products made by each glucosyltransferase
when reacted with Reb A and the specified sugar donor.

| | | | Products | | | | | | | | | | | | |
|---|---|---|---|---|---|---|---|---|---|---|---|---|---|---|---|
| | | | RebD Isomers | | | | RebM Isomers | | | | RebMp1 Isomers | | | | |
| Plasmid ID | Seq No | Sugar-Donor | 1.07 | 1.21 | 1.25 | 1.29 | 0.92 | 1.06 | 1.11 | 1.17 | 0.88 | 1.14 | 1.19 | 1.27 | 1.42 |
| pA10073 | 54 | Maltose | + | + | | + | | + | + | + | | | + | + | + |
| pA10074 | 49 | Maltose | + | + | | + | | | + | + | | | | | |
| pA10075 | 50 | Maltose | + | + | | + | | + | + | + | | | | + | + |
| pA10076 | 45 | Maltose | + | + | | + | | + | + | | | | | | |
| pA10078 | 53 | Maltose | | + | | + | | + | + | + | | | + | + | + |
| pA10151 | 44 | AG1P | + | | | | | | | | | | | | |
| pA10151 | 44 | Maltose | + | | | | | | | | | | | | |
| pA10151 | 44 | Sucrose | + | | + | | | | | | | | | | |
| pA10152 | 43 | AG1P | + | | | | | | | | | | | | |
| pA10152 | 43 | Maltose | + | | | | | | | | | | | | |
| pA10152 | 43 | Sucrose | + | | + | | + | | | | | | | | |
| pA10154 | 42 | AG1P | + | | | | + | | | | | | | | |
| pA10154 | 42 | Maltose | + | | | | | | | | | | | | |
| pA10154 | 42 | Sucrose | + | | | | + | | | | + | | | | |
| pA10155 | 46 | Maltose | + | | + | | | | | | | | | | |
| pA10174 | 41 | Maltose | | + | | + | | + | + | + | | + | + | + | + |
| pA10176 | 52 | Maltose | + | + | + | + | | | | | | | | | |
| pA10194 | 51 | Maltose | + | + | | + | | + | + | + | | | + | + | + |

Note that the RebMp1 relative retention times in Example 16 are calculated relative to the RebD retention time.

### Example 17: Rebaudioside Production From RebD Feed with Maltose and Alpha-Glucose-1-Phosphate Sugar Donor

The polypeptides encoded by the plasmids pA10073, pA10074, pA10075, pA10076, pA10078, pA10080, pA10082, pA10084, pA10109, pA10112, pA10117, pA10131, pA10158, pA10159, pA10160, pA10174, pA10175, pA10176, pA10189, pA10194, pA10195, pA10197, pA10207, pA10216, pA10259, pA10273 were expressed and purified as in Example 1 and 2. The enzymes were assayed in 100 mM of appropriate buffer (sodium acetate buffer, pH 5 ; HEPES buffer, pH 7.5) with 0.375 mM rebD, 5 mM sugar donor (maltose or alpha-glucose-1phosphate), 50 mM NaCl, 1 mM of appropriate co-factor (MnCl₂ or CaCl₂) and 1% DMSO overnight at 30° C. Enzymatic reactions were analyzed for rebaudioside content as in Example 16. Glucosyltransferase activity for rebD isomers, rebM isomers, and rebMp1 isomers are shown in Table 8.

TABLE 8
Summary of steviol glycoside products made by each glucosyltransferase
when reacted with Reb D and the specified sugar donor.

| | | | Products | | | | | | |
|---|---|---|---|---|---|---|---|---|---|
| | | | RebM Isomers | | | RebMp1 Isomers | | | |
| Plasmid ID | SeqNo | Sugar-Donor | 0.66 | 0.80 | 0.92 | 0.62 | 0.76 | 0.82 | 0.94 |
| pA10073 | 54 | Maltose | + | + | + | + | + | + | + |
| pA10075 | 50 | Maltose | + | + | + | + | + | + | + |
| pA10076 | 45 | Maltose | + | + | + | | | | |
| pA10078 | 53 | Maltose | + | + | + | + | + | + | + |
| pA10112 | 109 | Maltose | + | + | + | | | | |
| pA10158 | 56 | AG1P | | | | + | | | |
| pA10174 | 41 | Maltose | + | | | + | | | |
| pA10175 | 111 | Maltose | + | + | + | + | | | |
| pA10176 | 52 | Maltose | + | + | + | | | | |
| pA10194 | 51 | Maltose | + | + | + | + | + | | + |
| pA10195 | 63 | Maltose | + | | | + | | | |
| pA10197 | 62 | Maltose | + | | | + | | | |

Note that the RebMp1 relative retention times in Example 17 are calculated relative to the RebD retention time.

### Example 18: Rebaudioside Production with RebA 50% feed with Maltose and Sucrose Sugar Donors

The polypeptides encoded by the plasmids pA10074, pA10075, pA10076, pA10151, pA10152, pA10154, pA10176, pA10073, pA10078, pA10155, pA10174, pA10194, pA10114, pA12546 were expressed and purified as in Example 1 and 2. The enzymes were assayed for rebaudioside production in 100 mM of appropriate buffer (sodium acetate buffer, pH 5; HEPES buffer, pH 7.5) with 1 mg/mL 50% RebA (RA50), 10 mM sugar donor (maltose or sucrose), 50 mM NaCl, 1 mM CaCl₂ and 0.8% DMSO overnight at 30° C. To measure rebaudioside products, enzyme solutions were diluted to a final concentration of 30% DMSO and were further diluted 1:10 in water. Quantification was performed on an Agilent 6470 Triple quad mass spectrometer using an Agilent 1290 infinity II LC system with binary pump and autosampler (Table 9). A 5 min gradient was used and data was collected using a multi-reaction monitoring (MRM) quantification method specifically identifying the 1127→803 (reb-D), 965→803 (reb-A), and 1289→803 (reb-M)/1289→965 (reb-M isomer) transitions. Measured amounts of RebD_107, RebD_121 and RebD_129 are shown in Table 10.

TABLE 9
Instrument Settings

| | |
|---|---|
| Column | Waters Acquity UPLC HSS T3 1.8 um. 100 × 50 mm |
| | (p/n 186003539) |
| Temp | 40 C. |
| Flow rate | 0.45 ml/min |
| Injection | 1 uL |
| Solvents | A = 0.1% formic acid in water. B = 0.1% formic |
| | in acetonitrile |
| Wash | 30% Methanol |

### Example 19: Scaled-Up Production of RebD_107 with pA10154

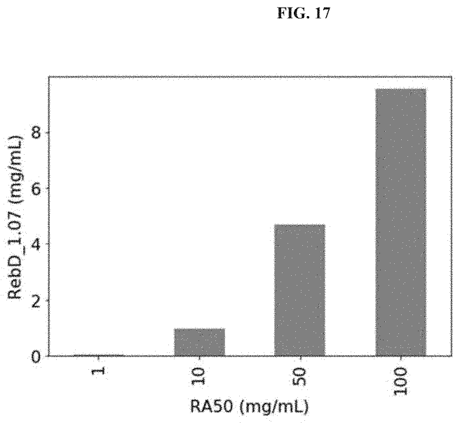

The polypeptide encoded by plasmid pA10154 (SEQ ID NO: 42) was expressed as in Example 1. The cells were lysed with lysis buffer and the enzyme was purified with gravity-flow immobilized metal affinity chromatography (IMAC). In addition, the microorganism containing the pA10154 plasmid was grown in a 1L fermentation. Ten grams of cells from the fermentation were lysed by French Press and the expressed enzyme was purified by gravity flow IMAC. The pA10154 enzyme was assayed for rebaudioside production in 100 mM sodium acetate buffer, pH 5 with 50 mM NaCl and 1 mM CaCl₂ overnight at 30° C. Rebaudioside conversion with four RA50 concentrations were evaluated (1, 10, 50 or 100 mg/mL RA50) with the corresponding sucrose concentrations: 10, 100, 500 and 1000 mM. The rebaudioside products were measured the same as Example 18. To calculate RebD_1.07 concentrations, the RebD_1.07 response was taken to be the same as the RebD standard response. Absolute yield of RebD_1.07 increases with increasing RA50 (FIG. 17)

TABLE 10
Measured area for RebD isomers RebD_1.07,
RebD_1.21 and RebD_1.29

| Plasmid | | Sugar | RebD Isomer | | |
|---|---|---|---|---|---|
| ID | PH | Donor | 1.07 | 1.21 | 1.29 |
| pA10073 | 7.5 | Maltose | 56363 | 128280 | 518454 |
| pA10075 | 7.5 | Maltose | 6035 | 173703 | 487504 |
| pA10174 | 7.5 | Maltose | 22270 | 73167 | 534386 |
| pA10194 | 7.5 | Maltose | 3517 | 164035 | 419451 |
| pA10078 | 7.5 | Maltose | 11187 | 27962 | 521876 |
| pA10114 | 7.5 | Maltose | 6767 | 141204 | 411987 |
| pA10154 | 5 | Sucrose | 549795 | 202 | 1097 |
| pA10154 | 7.5 | Maltose | 873 | 76412 | 420921 |
| pA10174 | 5 | Maltose | 472 | 54553 | 390157 |
| pA10194 | 5 | Maltose | 1212 | 96461 | 306866 |
| pA10114 | 5 | Maltose | 3711 | 99830 | 271071 |
| pA10073 | 5 | Maltose | 694 | 80564 | 288061 |
| pA10076 | 7.5 | Maltose | 30942 | 172786 | 89430 |
| pA10074 | 7.5 | Maltose | 5469 | 84134 | 199044 |
| pA10075 | 5 | Maltose | 2594 | 77070 | 206001 |
| pA10176 | 7.5 | Maltose | 4618 | 50702 | 227287 |
| pA10154 | 5 | Maltose | 3063 | 59282 | 203202 |
| pA10076 | 5 | Maltose | 13580 | 91103 | 56332 |
| pA10152 | 5 | Sucrose | 156177 | 837 | 853 |
| pA10074 | 5 | Maltose | 2158 | 38041 | 95052 |
| pA10176 | 5 | Maltose | 3098 | 20041 | 74062 |
| pA10155 | 7.5 | Maltose | 59116 | 606 | 1402 |
| pA10151 | 5 | Sucrose | 40464 | 5904 | 5845 |
| pA10151 | 5 | Maltose | 28324 | 444 | 2153 |
| pA10151 | 7.5 | Maltose | 4957 | 4370 | 5597 |
| pA10152 | 5 | Maltose | 10537 | 2346 | 848 |
| pA10078 | 5 | Maltose | 457 | 1662 | 9264 |
| pA10152 | 7.5 | Maltose | 1191 | 1226 | 3009 |
| pA12546 | 7.5 | Maltose | 347 | 639 | 2335 |
| pA12546 | 5 | Maltose | 1066 | 541 | 1613 |
| pA10155 | 5 | Maltose | 1269 | 229 | 299 |

### Example 20: Production of Reb M Isomers with RebD

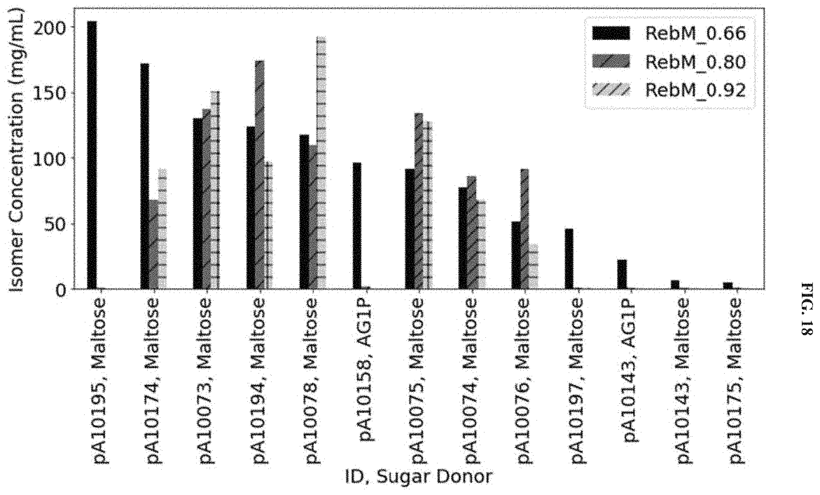

The polypeptides encoded by the plasmids pA10073, pA10078, pA10084, pA10112, pA10117, pA10159, pA10160, pA10174, pA10175, pA10194, pA10195, pA10197, pA10216, pA10273 were expressed and purified as in Example 1 and 2. The enzymes were assayed for rebaudioside production in 100 mM of HEPES buffer, pH 7.5 with 0.75 mM RebD, 5 mM sugar donor (maltose or alpha-glucose-1-phosphate), 50 mM NaCl, 1 mM CaCl₂ and 2% DMSO overnight at 30° C. The rebaudioside products were measured the same as Example 18. The analysis specifically focused on identifying the amount of RebM_0.66, RebM_0.80, and RebM_0.92. The measured conversion for each enzyme is shown in FIG. 18.

1. A method for transferring a sugar moiety to a substrate steviol glycoside, the method comprising contacting the substrate steviol glycoside with a glycosyltransferase polypeptide and a non-UDP-sugar sugar donor.

2. The method of claim 1, wherein the glycosyltransferase polypeptide comprises an amino acid sequence that is at least 60%, at least 65%, at least 70%, at least 75%, at least 80%, at least 85%, at least 90%, at least 95%, at least 98%, or at least 99% identical to an amino acid sequence selected from the group consisting of SEQ ID NOs: 1-128.

3. The method of claim 1 or claim 2, wherein the glycosyltransferase polypeptide comprises an amino acid sequence selected from the group consisting of SEQ ID NOs: 1-128.

4. The method of any one of claims 1-3, wherein the glycosyltransferase polypeptide comprises an amino acid sequence selected from the group consisting of SEQ ID NOs: 1, 19, 29, 32, 41, 42, 43, 44, 45, 46, 47, 48, 49, 50, 51, 52, 53, 54, 55, 56, 58, 61, 65, 67, 68, 81, 87, 90, 91, 92, 93, 94, 95, 96, 97, 98, 99, 100, 101, 102, 105, 106, 116, 120, 122, and 127.

5. The method of any one of claims 1-3, wherein the glycosyltransferase polypeptide comprises an amino acid sequence selected from the group consisting of SEQ ID NOs: 2, 3, 22, 29, 39, 41, 42, 45, 49, 50, 51, 52, 53, 54, 55, 56, 62, 63, 72, 87, 90, 91, 94, 97, 98, 99, 100, 101, 102, 109, 110, 111, 112, 113, 115, 117, 118, 119, 121, 123, 124, 125, 126, and 128.

6. The method of any one of claims 1-5, wherein the substrate steviol glycoside is steviol, steviol-13-O-glucoside, steviol-19-O-glucoside, rubusoside, steviol-1,2-bioside, steviol-1,3-bioside, rubusoside, dulcoside B, dulcoside A, rebaudioside B, rebaudioside G, stevioside, rebaudioside C, rebaudioside F, rebaudioside A, rebaudioside I, rebaudioside E, rebaudioside H, rebaudioside L, rebaudioside K, rebaudioside J, rebaudioside M, rebaudioside D, rebaudioside N, rebaudioside O, rebaudioside Q, an isomer thereof, a synthetic steviol glycoside or combinations thereof.

7. The method of any one of claims 1-6, wherein the substrate steviol glycoside is rebaudioside A, an isomer thereof, or combinations thereof.

8. The method of any one of claims 1-6, wherein the substrate steviol glycoside is rebaudioside D, an isomer thereof, or combinations thereof.

9. The method of any one of claims 1-8, wherein the non-UDP-sugar sugar donor is alpha-glucose-1-phosphate, beta-glucose-1-phosphate, cellobiose, sucrose, maltose, gentiobiose, trehalose, kojibiose, nigerose, isomaltose, beta-beta-trehalose, alpha-beta-trehalose, sophorose, laminaribiose, turanose, maltulose, palatinose, gentiobiulose, nigerotriose, maltotriose, melezitose, maltotriulose, kestose, starch, cellulose, glycogen, amylose, amylopectin, dextran, dextrin, maltodextrin, glucose syrup, cellodextrin, cyclodextrin, a non-UDP nucleotide sugar (i.e. ADP-glucose, GDP-glucose, CDP-glucose, TDP-glucose), or a steviol glycoside.

10. The method of any one of claims 1-8, wherein the non-UDP-sugar sugar donor is not alpha-glucose-1-phosphate.

11. The method of any one of claims 1-8, wherein the non-UDP-sugar sugar donor is not beta-glucose-1-phosphate.

12. The method of any one of claims 1-11, wherein the glucosyltransferase polypeptide is expressed in a host microorganism.

13. The method of claim 12, wherein the host microorganism is E. coli, Saccharomyces sp., Aspergillus sp., Pichia sp., or Bacillus sp.

14. The method of any one of claims 1-13, wherein the glucosyltransferase polypeptide is immobilized to a solid support.

15. The method of claim 14, wherein the solid support is derivatized cellulose, glass, ceramic, methacrylate, styrene, acrylic, a metal oxide, or a membrane.

16. The method of claim 14 or claim 15, wherein the glucosyltransferase polypeptide is immobilized to the solid support by covalent attachment, adsorption, cross-linking, entrapment, or encapsulation.

17. The method of any one of claims 1-16, wherein the contacting the substrate steviol glycoside with the glycosyltransferase polypeptide and the non-UDP-sugar sugar donor is in a reaction medium comprising pyridoxal phosphate (PLP) and/or one or more metal ions.

18. The method of any one of claims 1-17, wherein the contacting the substrate steviol glycoside with the glycosyltransferase polypeptide and the non-UDP-sugar sugar donor is at temperature between about 0° C. and about 60° C.

19. The method of any one of claims 1-18, wherein the contacting the substrate steviol glycoside with the glycosyltransferase polypeptide and the non-UDP-sugar sugar donor is at temperature about 30° C.

20. The method of any one of claims 1-19, wherein the contacting the substrate steviol glycoside with the glycosyltransferase polypeptide and the non-UDP-sugar sugar donor is carried out in a duration of time between 1 hour and 1 week.

21. The method of any one of claims 1-20, wherein the contacting the substrate steviol glycoside with the glycosyltransferase polypeptide and the non-UDP-sugar sugar donor is carried out in a duration of time of about 24 hours.

22. A method for producing a target steviol glycoside composition, comprising the steps of:
(a) providing a starting composition comprising greater than about 0.5%, about 1%, about 2%, about 3%, about 4%, about 5%, about 10%, about 20%, about 30%, about 40%, about 50%, about 60%, about 70%, about 80%, about 90%, about 92%, about 93%, about 94%, about 95%, about 96%, about 97%, about 98%, about 99%, or about 99.6% of a substrate steviol glycoside by weight on an anhydrous basis;
(b) contacting the starting composition with a glycosyltransferase polypeptide and a non-UDP-sugar sugar donor; and
(c) producing a target composition comprising a target steviol glycoside.

23. The method of claim 22, wherein the glycosyltransferase polypeptide comprises an amino acid sequence that is at least 60%, at least 65%, at least 70%, at least 75%, at least 80%, at least 85%, at least 90%, at least 95%, at least 98%, or at least 99% identical to an amino acid sequence selected from the group consisting of SEQ ID NOs: 1-128.

24. The method of claim 22 or claim 23, wherein the target composition comprises greater than about 0.5%, about 1%, about 2%, about 3%, about 4%, about 5%, about 10%, about 20%, about 30%, about 40%, about 50%, about 60%, about 70%, about 80%, about 85%, about 90%, about 91%, about 92%, about 93%, about 94%, about 95%, about 96%, about 97%, about 98%, about 99%, about 99.6% of the target steviol glycoside by weight on an anhydrous basis.

25. The method of any one of claims 22-24, wherein the substrate steviol glycoside is steviol, steviol-13-O-glucoside, steviol-19-O-glucoside, rubusoside, steviol-1,2-bioside, steviol1,3-bioside, rubusoside, dulcoside B, dulcoside A, rebaudioside B, rebaudioside G, stevioside, rebaudioside C, rebaudioside F, rebaudioside A, rebaudioside I, rebaudioside E, rebaudioside H, rebaudioside L, rebaudioside K, rebaudioside J, rebaudioside M, rebaudioside D, rebaudioside N, rebaudioside O, rebaudioside Q, an isomer thereof, a synthetic steviol glycoside or combinations thereof.

26. The method of any one of claims 22-25, wherein the non-UDP-sugar sugar donor is alpha-glucose-1-phosphate, beta-glucose-1-phosphate, cellobiose, sucrose, maltose, gentiobiose, trehalose, kojibiose, nigerose, isomaltose, beta-beta-trehalose, alpha-beta-trehalose, sophorose, laminaribiose, turanose, maltulose, palatinose, gentiobiulose, nigerotriose, maltotriose, melezitose, maltotriulose, kestose, starch, cellulose, glycogen, amylose, amylopectin, dextran, dextrin, maltodextrin, glucose syrup, cellodextrin, cyclodextrin, a non-UDP nucleotide sugar (i.e. ADP-glucose, GDP-glucose, CDP-glucose, TDP-glucose), or a steviol glycoside.

27. The method of any one of claims 22-25, wherein the non-UDP-sugar sugar donor is not alpha-glucose-1-phosphate.

28. The method of any one of claims 22-25, wherein the non-UDP-sugar sugar donor is not beta-glucose-1-phosphate.

29. The method of any one of claims 22-28, wherein at least about 5%, about 10%, about 20%, about 30%, about 40%, about 50%, about 60%, about 70%, about 80%, or about 90% of the substrate steviol glycoside in the starting composition is converted to the target steviol glycoside.

30. The method of any one of claims 22-29, wherein the substrate steviol glycoside is rebaudioside A, or isomers thereof; and the target steviol glycoside is rebaudioside D, or isomers thereof

31. The method of claim 30, wherein the target steviol glycoside is a rebaudioside D isomer.

32. The method of claim 30, wherein the rebaudioside D isomer is rebaudioside D_1.07, rebaudioside D_0.77, rebaudioside D_1.21, rebaudioside D_1.25, or rebaudioside D_ 1.29.

33. The method of any one of claims 22-29, wherein the substrate steviol glycoside is rebaudioside A, or isomers thereof; and the target steviol glycoside is rebaudioside M, or isomers thereof

34. The method of claim 33, wherein the target steviol glycoside is a rebaudioside M isomer.

35. The method of claim 34, wherein the rebaudioside M isomer is rebaudioside M_0.66, rebaudioside M_0.80, rebaudioside M_0.92, rebaudioside M_1.06, rebaudioside M_1.11, rebaudioside M_1.17, or rebaudioside M_1.51.

36. The method of any one of claims 22-29, wherein the substrate steviol glycoside is rebaudioside A, or isomers thereof; and the target steviol glycoside is a rebaudioside Mp1 isomer.

37. The method of claim 36, wherein the rebaudioside Mp1 isomer is rebaudioside Mp1_0.62, rebaudioside Mp1_0.76, rebaudioside Mp1_0.82, rebaudioside Mp1_0.88, rebaudioside Mp1_0.94, rebaudioside Mp1_1.09, rebaudioside Mp1_1.14, rebaudioside Mp1_1.19, rebaudioside Mp1_1.27, rebaudioside Mp1_1.42, or rebaudioside Mp1_1.66.

38. The method of any one of claims 22-29, wherein the substrate steviol glycoside is rebaudioside D, or isomers thereof; and the target steviol glycoside is rebaudioside M, or isomers thereof

39. The method of claim 38, wherein the substrate steviol glycoside is a rebaudioside D isomer.

40. The method of claim 39, wherein the rebaudioside D isomer is rebaudioside D_1.07, rebaudioside D_0.77, rebaudioside D_1.21, rebaudioside D_1.25, or rebaudioside D_1.29.

41. The method of any one of claims 38-40, wherein the target steviol glycoside is a rebaudioside M isomer.

42. The method of claim 41, wherein the rebaudioside M isomer is rebaudioside M_0.66, rebaudioside M_0.80, rebaudioside M_0.92, rebaudioside M_1.06, rebaudioside M_1.11, rebaudioside M_1.17, or rebaudioside M_1.51.

43. The method of any one of claims 22-42, wherein the starting composition is a Stevia rebaudiana extract.

44. The method of any one of claims 22-43, wherein the glucosyltransferase polypeptide is expressed in a host microorganism.

45. The method of claim 44, wherein the host microorganism is E. coli, Saccharomyces sp., Aspergillus sp., Pichia sp., or Bacillus sp.

46. The method of any one of claims 22-45, wherein the glucosyltransferase polypeptide is immobilized to a solid support.

47. The method of claim 46, wherein the solid support is derivatized cellulose, glass, ceramic, methacrylate, styrene, acrylic, a metal oxide, or a membrane.

48. The method of any one of claims 22-47, wherein the step of contacting the starting composition with the glycosyltransferase polypeptide and the non-UDP-sugar sugar donor is in a reaction medium comprising pyridoxal phosphate (PLP) and/or one or more metal ions.

49. The method of any one of claims 22-48, wherein the step of contacting the starting composition with the glycosyltransferase polypeptide and the non-UDP-sugar sugar donor is at temperature between about 0° C. and about 60° C.

50. The method of any one of claims 22-49, wherein the step of contacting the starting composition with the glycosyltransferase polypeptide and the non-UDP-sugar sugar donor is at temperature about 30° C.

51. The method of any one of claims 22-50, wherein the step of contacting the starting composition with the glycosyltransferase polypeptide and the non-UDP-sugar sugar donor is carried out in a duration of time between 1 hour and 1 week.

52. The method of any one of claims 22-51, wherein the step of contacting the starting composition with the glycosyltransferase polypeptide and the non-UDP-sugar sugar donor is carried out in a duration of time of about 24 hours.

53. A method for producing a target steviol glycoside composition, comprising the steps of:
(a) providing a starting composition comprising greater than about 0.5%, about 1%, about 2%, about 3%, about 4%, about 5%, about 10%, about 20%, about 30%, about 40%, about 50%, about 60%, about 70%, about 80%, about 90%, about 92%, about 93%, about 94%, about 95%, about 96%, about 97%, about 98%, about 99%, or about 99.6% of a substrate steviol glycoside by weight on an anhydrous basis;
(b) contacting the starting composition with a first glycosyltransferase polypeptide and a non-UDP-sugar sugar donor;
(c) producing an intermediate composition comprising an intermediate target steviol glycoside;
(d) contacting the intermediate composition with a second glycosyltransferase polypeptide and the non-UDP-sugar sugar donor; and
(e) producing a target composition comprising a target steviol glycoside.

54. The method of claim 53, wherein the first glycosyltransferase polypeptide comprises an amino acid sequence that is at least 60%, at least 65%, at least 70%, at least 75%, at least 80%, at least 85%, at least 90%, at least 95%, at least 98%, or at least 99% identical to an amino acid sequence selected from the group consisting of SEQ ID NOs: 1-128.

55. The method of claim 53 or claim 54, wherein the second glycosyltransferase polypeptide comprises an amino acid sequence that is at least 60%, at least 65%, at least 70%, at least 75%, at least 80%, at least 85%, at least 90%, at least 95%, at least 98%, or at least 99% identical to an amino acid sequence selected from the group consisting of SEQ ID NOs: 1-128.

56. The method of any one of claims 53-55, wherein the first glycosyltransferase polypeptide and the second glycosyltransferase polypeptide are identical.

57. The method of any one of claims 53-55, wherein the first glycosyltransferase polypeptide and the second glycosyltransferase polypeptide are different.

58. The method of any one of claims 53-57, wherein the intermediate composition comprises greater than about 5%, about 10%, about 20%, about 30%, about 40%, about 50%, about 60%, about 70%, about 80%, about 85%, about 90%, about 91%, about 92%, about 93%, about 94%, about 95%, about 96%, about 97%, about 98%, about 99%, or about 99.6% of the intermediate target steviol glycoside by weight on an anhydrous basis.

59. The method of any one of claims 53-58, wherein the target composition comprises greater than about 0.5%, about 1%, about 2%, about 3%, about 4%, about 5%, about 10%, about 20%, about 30%, about 40%, about 50%, about 60%, about 70%, about 80%, about 85%, about 90%, about 91%, about 92%, about 93%, about 94%, about 95%, about 96%, about 97%, about 98%, about 99%, or about 99.6% of the target steviol glycoside by weight on an anhydrous basis.

60. The method of any one of claims 53-59, wherein the substrate steviol glycoside is steviol, steviol-13-O-glucoside, steviol-19-O-glucoside, rubusoside, steviol-1,2-bioside, steviol1,3-bioside, rubusoside, dulcoside B, dulcoside A, rebaudioside B, rebaudioside G, stevioside, rebaudioside C, rebaudioside F, rebaudioside A, rebaudioside I, rebaudioside E, rebaudioside H, rebaudioside L, rebaudioside K, rebaudioside J, rebaudioside M, rebaudioside D, rebaudioside N, rebaudioside O, rebaudioside Q, an isomer thereof, a synthetic steviol glycoside or combinations thereof.

61. The method of any one of claims 53-60, the non-UDP-sugar sugar donor is alpha-glucose-1-phosphate, beta-glucose-1-phosphate, cellobiose, sucrose, maltose, gentiobiose, trehalose, kojibiose, nigerose, isomaltose, beta-beta-trehalose, alpha-beta-trehalose, sophorose, laminaribiose, turanose, maltulose, palatinose, gentiobiulose, nigerotriose, maltotriose, melezitose, maltotriulose, kestose, starch, cellulose, glycogen, amylose, amylopectin, dextran, dextrin, maltodextrin, glucose syrup, cellodextrin, cyclodextrin, a non-UDP nucleotide sugar (i.e. ADP-glucose, GDP-glucose, CDP-glucose, TDP-glucose), or a steviol glycoside.

62. The method of any one of claims 53-61, wherein the non-UDP-sugar sugar donor is not alpha-glucose-1-phosphate.

63. The method of any one of claims 53-62, wherein the non-UDP-sugar sugar donor is not beta-glucose-1-phosphate.

64. The method of any one of claims 53-63, wherein at least about 5%, about 10%, about 20%, about 30%, about 40%, about 50%, about 60%, about 70%, about 80%, or about 90% of the substrate steviol glycoside in the starting composition is converted to the intermediate target steviol glycoside.

65. The method of any one of claims 53-64, wherein at least about 5%, about 10%, about 20%, about 30%, about 40%, about 50%, about 60%, about 70%, about 80%, or about 90% of the intermediate target steviol glycoside in the intermediate composition is converted to the target steviol glycoside.

66. The method of any one of claims 53-65, wherein the substrate steviol glycoside is rebaudioside A, or isomers thereof; and the intermediate steviol glycoside is rebaudioside D, or isomers thereof; and the target steviol glycoside is rebaudioside M, or isomers thereof.

67. The method of any one of claims 53-66, wherein the starting composition is a Stevia rebaudiana extract.

68. The method of any one of claims 53-67, wherein the glucosyltransferase polypeptide is expressed in a host microorganism.

69. The method of claim 68, wherein the host microorganism is E. coli, Saccharomyces sp., Aspergillus sp., Pichia sp., or Bacillus sp.

70. The method of any one of claims 53-69, wherein the glucosyltransferase polypeptide is immobilized to a solid support.

71. The method of claim 70, wherein the solid supports is derivatized cellulose or glass, ceramics, methacrylate, styrene, acrylic, metal oxides, or membranes.

72. The method of any one of claims 53-71, wherein the step of contacting the starting composition with the first glycosyltransferase polypeptide and the non-UDP-sugar sugar donor is in a reaction medium comprising pyridoxal phosphate (PLP) and/or one or more metal ions.

73. The method of any one of claims 53-72, wherein the step of contacting the starting composition with the first glycosyltransferase polypeptide and the non-UDP-sugar sugar donor is at temperature between about 0° C. and about 60° C.

74. The method of any one of claims 53-73, wherein the step of contacting the starting composition with the first glycosyltransferase polypeptide and the non-UDP-sugar sugar donor is at temperature about 30° C.

75. The method of any one of claims 53-74, wherein the step of contacting the starting composition with the first glycosyltransferase polypeptide and the non-UDP-sugar sugar donor is carried out in a duration of time between 1 hour and 1 week.

76. The method of any one of claims 53-75, wherein the step of contacting the starting composition with the first glycosyltransferase polypeptide and the non-UDP-sugar sugar donor is carried out in a duration of time of about 24 hours.

77. The method of any one of claims 53-76, wherein the step of contacting the intermediate composition with the second glycosyltransferase polypeptide and the non-UDP-sugar sugar donor is in a reaction medium comprising pyridoxal phosphate (PLP) and/or one or more metal ions.

78. The method of any one of claims 53-77, wherein the step of contacting the intermediate composition with the second glycosyltransferase polypeptide and the non-UDP-sugar sugar donor is at temperature between about 0° C. and about 60° C.

79. The method of any one of claims 53-78, wherein the step of contacting the intermediate composition with the second glycosyltransferase polypeptide and the non-UDP-sugar sugar donor is at temperature about 30° C.

80. The method of any one of claims 53-79, wherein the step of contacting the intermediate composition with the second glycosyltransferase polypeptide and the non-UDP-sugar sugar donor is carried out in a duration of time between 1 hour and 1 week.

81. The method of any one of claims 53-80, wherein the step of contacting the intermediate composition with the second glycosyltransferase polypeptide and the non-UDP-sugar sugar donor is carried out in a duration of time of about 24 hours.

82. A recombinant glycosyltransferase polypeptide comprising an amino acid sequence that is at least 60%, at least 65%, at least 70%, at least 75%, at least 80%, at least 85%, at least 90%, at least 95%, at least 98%, or at least 99% identical to an amino acid sequence selected from the group consisting of SEQ ID NOs: 1-128.

83. The recombinant glycosyltransferase polypeptide of claim 82, wherein the glycosyltransferase polypeptide comprises an amino acid sequence that is at least 60% identical to identical to an amino acid sequence selected from the group consisting of SEQ ID NOs: 1, 19, 29, 32, 41, 42, 43, 44, 45, 46, 47, 48, 49, 50, 51, 52, 53, 54, 55, 56, 58, 61, 65, 67, 68, 81, 87, 90, 91, 92, 93, 94, 95, 96, 97, 98, 99, 100, 101, 102, 105, 106, 116, 120, 122, and 127.

84. The recombinant glycosyltransferase polypeptide of claim 82, wherein the glycosyltransferase polypeptide comprises an amino acid sequence that is at least 60% identical to identical to an amino acid sequence selected from the group consisting of SEQ ID NOs: 2, 3, 22, 29, 39, 41, 42, 45, 49, 50, 51, 52, 53, 54, 55, 56, 62, 63, 72, 87, 90, 91, 94, 97, 98, 99, 100, 101, 102, 109, 110, 111, 112, 113, 115, 117, 118, 119, 121, 123, 124, 125, 126, and 128.

85. The recombinant glycosyltransferase polypeptide of claim 82, wherein the glycosyltransferase polypeptide comprises an amino acid sequence selected from the group consisting of SEQ ID NOs: 1-128.

86. The recombinant glycosyltransferase polypeptide of claim 82 or claim 83, wherein the glycosyltransferase polypeptide comprises an amino acid sequence selected from the group consisting of SEQ ID NOs: 1, 19, 29, 32, 41, 42, 43, 44, 45, 46, 47, 48, 49, 50, 51, 52, 53, 54, 55, 56, 58, 61, 65, 67, 68, 81, 87, 90, 91, 92, 93, 94, 95, 96, 97, 98, 99, 100, 101, 102, 105, 106, 116, 120, 122, and 127.

87. The recombinant glycosyltransferase polypeptide of claim 82 or claim 84, wherein the glycosyltransferase polypeptide comprises an amino acid sequence selected from the group consisting of SEQ ID NOs: 2, 3, 22, 29, 39, 41, 42, 45, 49, 50, 51, 52, 53, 54, 55, 56, 62, 63, 72, 87, 90, 91, 94, 97, 98, 99, 100, 101, 102, 109, 110, 111, 112, 113, 115, 117, 118, 119, 121, 123, 124, 125, 126, and 128.

88. The recombinant glycosyltransferase polypeptide of any one of claims 82-87, wherein the glycosyltransferase polypeptide is capable of transferring a sugar moiety to a substrate steviol glycoside.

89. The recombinant glycosyltransferase polypeptide of claim 88, wherein the substrate steviol glycoside is steviol, steviol-13-O-glucoside, steviol-19-O-glucoside, rubusoside, steviol-1,2-bioside, steviol-1,3-bioside, rubusoside, dulcoside B, dulcoside A, rebaudioside B, rebaudioside G, stevioside, rebaudioside C, rebaudioside F, rebaudioside A, rebaudioside I, rebaudioside E, rebaudioside H, rebaudioside L, rebaudioside K, rebaudioside J, rebaudioside M, rebaudioside D, rebaudioside N, rebaudioside O, rebaudioside Q, an isomer thereof, a synthetic steviol glycoside or combinations thereof

90. The recombinant glycosyltransferase polypeptide of claim 88 or claim 89, wherein the substrate steviol glycoside is rebaudioside A, or an isomer thereof

91. The recombinant glycosyltransferase polypeptide of claim 88 or claim 89, wherein the substrate steviol glycoside is rebaudioside D, or an isomer thereof

92. The recombinant glycosyltransferase polypeptide of any one of claims 82-91, wherein the non-UDP-sugar sugar donor is alpha-glucose-1-phosphate, beta-glucose-1-phosphate, cellobiose, sucrose, maltose, gentiobiose, trehalose, kojibiose, nigerose, isomaltose, beta-beta-trehalose, alpha-beta-trehalose, sophorose, laminaribiose, turanose, maltulose, palatinose, gentiobiulose, nigerotriose, maltotriose, melezitose, maltotriulose, kestose, starch, cellulose, glycogen, amylose, amylopectin, dextran, dextrin, maltodextrin, glucose syrup, cellodextrin, cyclodextrin, a non-UDP nucleotide sugar (i.e. ADP-glucose, GDP-glucose, CDP-glucose, TDP-glucose), or a steviol glycoside.

93. The recombinant glycosyltransferase polypeptide of any one of claims 82-92, wherein the glucosyltransferase polypeptide is expressed in a host microorganism.

94. The recombinant glycosyltransferase polypeptide of claim 93, wherein the host microorganism is E. coli, Saccharomyces sp., Aspergillus sp., Pichia sp., or Bacillus sp.

95. The recombinant glycosyltransferase polypeptide of any one of claims 82-92, wherein the glucosyltransferase polypeptide is immobilized to a solid support.

96. The recombinant glycosyltransferase polypeptide of claim 95, wherein the solid supports is derivatized cellulose, glass, ceramic, methacrylate, styrene, acrylic, a metal oxide, or a membrane.

97. A modified microorganism expressing a glycosyltransferase polypeptide comprises an amino acid sequence that is at least 60%, at least 65%, at least 70%, at least 75%, at least 80%, at least 85%, at least 90%, at least 95%, at least 98%, or at least 99% identical to an amino acid sequence selected from the group consisting of SEQ ID NOs: 1-128.

98. The modified microorganism of claim 97, wherein the glycosyltransferase polypeptide comprises an amino acid sequence that is at least 60% identical to an amino acid sequence selected from the group consisting of SEQ ID NOs: 1, 19, 29, 32, 41, 42, 43, 44, 45, 46, 47, 48, 49, 50, 51, 52, 53, 54, 55, 56, 58, 61, 65, 67, 68, 81, 87, 90, 91, 92, 93, 94, 95, 96, 97, 98, 99, 100, 101, 102, 105, 106, 116, 120, 122, and 127.

99. The modified microorganism of claim 97, wherein the glycosyltransferase polypeptide comprises an amino acid sequence that is at least 60% identical to an amino acid sequence selected from the group consisting of SEQ ID NOs: 2, 3, 22, 29, 39, 41, 42, 45, 49, 50, 51, 52, 53, 54, 55, 56, 62, 63, 72, 87, 90, 91, 94, 97, 98, 99, 100, 101, 102, 109, 110, 111, 112, 113, 115, 117, 118, 119, 121, 123, 124, 125, 126, and 128.

100. The modified microorganism of claim 97, wherein the glycosyltransferase polypeptide comprises an amino acid sequence selected from the group consisting of SEQ ID NOs: 1-128.

101. The modified microorganism of claim 97 or claim 98, wherein the glycosyltransferase polypeptide comprises an amino acid sequence selected from the group consisting of SEQ ID NOs: 1, 19, 29, 32, 41, 42, 43, 44, 45, 46, 47, 48, 49, 50, 51, 52, 53, 54, 55, 56, 58, 61, 65, 67, 68, 81, 87, 90, 91, 92, 93, 94, 95, 96, 97, 98, 99, 100, 101, 102, 105, 106, 116, 120, 122, and 127

102. The modified microorganism of claim 97 or claim 99, wherein the glycosyltransferase polypeptide comprises an amino acid sequence selected from the group consisting of SEQ ID NOs: 2, 3, 22, 29, 39, 41, 42, 45, 49, 50, 51, 52, 53, 54, 55, 56, 62, 63, 72, 87, 90, 91, 94, 97, 98, 99, 100, 101, 102, 109, 110, 111, 112, 113, 115, 117, 118, 119, 121, 123, 124, 125, 126, and 128.

103. The modified microorganism of any one of claims 97-102, wherein the glycosyltransferase polypeptide is capable of transferring a sugar moiety to a substrate steviol glycoside.

104. The modified microorganism of claim 103, wherein the substrate steviol glycoside is steviol, steviol-13-O-glucoside, steviol-19-O-glucoside, rubusoside, steviol-1,2-bioside, steviol-1,3-bioside, rubusoside, dulcoside B, dulcoside A, rebaudioside B, rebaudioside G, stevioside, rebaudioside C, rebaudioside F, rebaudioside A, rebaudioside I, rebaudioside E, rebaudioside H, rebaudioside L, rebaudioside K, rebaudioside J, rebaudioside M, rebaudioside D, rebaudioside N, rebaudioside O, rebaudioside Q, an isomer thereof, a synthetic steviol glycoside or combinations thereof

105. The modified microorganism of claim 103 or claim 104, wherein the substrate steviol glycoside is rebaudioside A, or an isomer thereof.

106. The modified microorganism of claim 103 or claim 104, wherein the substrate steviol glycoside is rebaudioside D, or an isomer thereof.

107. The modified microorganism of any one of claims 97-106, wherein the non-UDPsugar sugar donor is alpha-glucose-1-phosphate, beta-glucose-1-phosphate, cellobiose, sucrose, maltose, gentiobiose, trehalose, kojibiose, nigerose, isomaltose, beta-beta-trehalose, alpha-beta-trehalose, sophorose, laminaribiose, turanose, maltulose, palatinose, gentiobiulose, nigerotriose, maltotriose, melezitose, maltotriulose, kestose, starch, cellulose, glycogen, amylose, amylopectin, dextran, dextrin, maltodextrin, glucose syrup, cellodextrin, cyclodextrin, a non-UDP nucleotide sugar (i.e. ADP-glucose, GDP-glucose, CDP-glucose, TDP-glucose), or a steviol glycoside.

108. The modified microorganism of any one of claims 97-107, wherein the modified microorganism is E. coli, Saccharomyces sp., Aspergillus sp., Pichia sp., or Bacillus sp.

109. The modified microorganism of any one of claims 97-108, wherein the glucosyltransferase polypeptide is immobilized to a solid support.

110. The modified microorganism of claim 109, wherein the solid supports is derivatized cellulose, glass, ceramic, methacrylate, styrene, acrylic, a metal oxide, or a membrane.
